(19) United States
(12) Patent Application Publication
(10) Pub. No.: US 2013/0309138 A1
(43) Pub. Date: Nov. 21, 2013

(54) Preservation System for Consumables and Cosmetic Substances

(71) Applicant: Eugenio Minvielle, Rye, NY (US)

(72) Inventor: Eugenio Minvielle, Rye, NY (US)

(21) Appl. No.: 13/948,083

(22) Filed: Jul. 22, 2013

Related U.S. Application Data
(63) Continuation-in-part of application No. 13/937,167, filed on Jul. 8, 2013.
Application No. 13/937,167 is a continuation-in-part of application No. 13/732,050, filed on Dec. 31, 2012.
Application No. 13/732,050 is a continuation-in-part of application No. 13/485,878, filed on May 31, 2012.
Application No. 13/485,878 is a continuation-in-part of application No. 13/888,353, filed on May 6, 2013.
Application No. 13/888,353 is a continuation-in-part of application No. 13/485,854, filed on May 31, 2012.
(60) Provisional application No. 61/624,800, filed on Apr. 16, 2012.
(60) Provisional application No. 61/624,980, filed on Apr. 16, 2012.
(60) Provisional application No. 61/624,989, filed on Apr. 16, 2012.
(60) Provisional application No. 61/624,948, filed on Apr. 16, 2012.
(60) Provisional application No. 61/624,972, filed on Apr. 16, 2012.
(60) Provisional application No. 61/624,985, filed on Apr. 16, 2012.

(51) Int. Cl. A61J 1/00; G01N 33/00

(52) U.S. Cl. CPC A61J 1/00; G01N 33/00
USPC 422/105; 73/865.8

(57) ABSTRACT

Disclosed herein is preservation system for consumables and cosmetic substances. The preservation system obtains information about the consumables and cosmetic substance to be preserved, senses and measures the external environment to the preservation system, senses and measures the internal environment to the preservation system, senses and measures the state of the consumables and cosmetic substance, and stores such information throughout the period of preservation. Using this accumulated information, the preservation system can measure, or estimate, changes in efficacy content (usually degradation) during the period of preservation. Additionally, the preservation system can use this information to dynamically modify the preservation system to minimize detrimental changes to the efficacy content of the consumables and cosmetic substance, and in some cases actually improve the consumables and cosmetic substance attributes.

### RELATED PATENT APPLICATIONS

This application is a continuation-in-part of Utility application U.S. Ser. No. 13/888,353 filed May 6, 2013, titled PRESERVATION SYSTEM FOR NUTRITIONAL SUBSTANCES,” which is a continuation-in-part of Utility application U.S. Ser. No. 13/485,854 filed May 31, 2012, which claims benefit under 35 U.S.C. 119(e) of U.S. Provisional Patent Application Ser. No. 61/624,948 filed Apr. 16, 2012; U.S. Provisional Patent Application Ser. No. 61/624,972 filed Apr. 16, 2012; and U.S. Provisional Patent Application 61/624,985 filed Apr. 16, 2012, the contents of which are all incorporated herein by reference in their entirety. This application is also a continuation-in-part of Utility application U.S. Ser. No. 13/937,167 filed Jul. 8, 2013, titled “CONSUMER INFORMATION AND SENSING SYSTEM FOR NUTRITIONAL SUBSTANCES,” which is a continuation-in-part of Utility application U.S. Ser. No. 13/732,050 filed Dec. 31, 2012, which is a continuation-in-part of Utility application U.S. Ser. No. 13/485,878 filed May 31, 2012, which claims priority to U.S. Provisional Patent Application Ser. No. 61/624,800, filed Apr. 16, 2012; U.S. Provisional Patent Application Ser. No. 61/624,980, filed Apr. 16, 2012; and U.S. Provisional Patent Application, 61/624,989, filed Apr. 16, 2012, the contents of which are all incorporated herein by reference in their entirety.

### FIELD OF THE INVENTION

The present inventions relate to collection, transmission, creation and use of information regarding the preservation of consumables and cosmetic substances.

### BACKGROUND OF THE INVENTION

Consumables and cosmetic substances comprise a wide array of products that are used by consumers to enhance their health, quality of life, hygiene, scent, appearance, and so forth. Consumables and cosmetic substances and their ingredients may be grown (plants), raised (animals) or synthesized (synthetic compounds), or transformed from any combination of consumables and cosmetic ingredients. Additionally, consumables and cosmetic substances can be sourced from, or found in a wild, non-cultivated form, which can be caught or collected. While the collectors and creators of consumables and cosmetic substances generally obtain and/or generate information about the source, history, and active ingredient content of their products, they generally do not pass such information along to the users of their products. One reason is that the participant groups in the consumables and cosmetic substance industry have tended to act like “silo” industries. Each group in the consumables and cosmetic substance industry: growers, collectors, packagers, processors, distributors, retailers, and compounders work separately, and either shares little to no information between themselves. There is generally no consumer access to, and little traceability of, information regarding the creation and/or origin, preservation, processing, preparation, dispensing, or local storage of consumables and cosmetic substances. Further, there is no information available to consumers as to the evolution of a consumables and cosmetic substance's efficacy. Such evolution in efficacy is typically a degradation, but could be a maintenance or improvement. It would be desirable for such information be available to the consumers of consumables and cosmetic substances, as well as all participants in the consumables and cosmetic industry—the consumables and cosmetic substance supply system.

While the consumables and cosmetic substances supply system has endeavored to increase the efficacy value of the consumables and cosmetic substances it produces, it has not provided a means of tracking the evolution of consumables and cosmetic substances efficacy value in a manner that makes information regarding the evolution available, useful, or responsive to consumers. The efficacy value of consumables and cosmetic substances refers to the portion of these consumables and cosmetic substances which are beneficial to the organisms which use them. Examples of the efficacy value of consumables and cosmetic substances could include: particular Aloe vera derived phytochemical concentrations in skin lotion; antibiotic concentration in antibiotic ointment; percent by weight of Naproxen in a pain pill; concentration of Omega oil in a dietary supplement; concentration of tea tree oil in a shampoo; concentration of tetrahydrozoline hydrochloride in eye drops; Vitamin C level in a vitamin pill; specific amino-acid protein levels in lip stick; amount of epinephrine in an emergency epinephrine injector; the concentration of Hydrogen Peroxide in a bottle of antiseptic; and an amount or concentration of any other component which is necessary, or at least beneficial, to the organism using the consumables and cosmetic substances.

While there has recently been greater attention by consumer organizations, health organizations and the public to the efficacy value of consumables and cosmetic substances, the consumables and cosmetic substance industry has been slow in responding to this attention. One reason for this may be that since the consumables and cosmetic substance industry operates as silos of those who create consumables and cosmetic substances, those who preserve and transport consumables and cosmetic substances, those who transform consumables and cosmetic substances, and those who finally prepare the consumables and cosmetic substances for use by the consumer, there has been no system wide coordination or management of efficacy value, and no practical way for creators, preservers, transformers, dispensers, and consumers to update labeling content for consumables and cosmetic substances. While each of these silo industries may be able to maintain or increase the efficacy value of the consumables and cosmetic substances they handle, each silo industry has only limited information and control of the consumables and cosmetic substances they receive, and the consumables and cosmetic substances they pass along, and the limited information in their control provides little utility beyond tracking product inventory and predetermined expiration dates.

As consumers better understand their need for consumables and cosmetic substances with higher efficacy value, they will start demanding that the consumables and cosmetic substance industry offer products which include higher efficacy value, and/or at least information regarding efficacy value of such products, as well as information regarding the source, creation and other origin information for the consumables and cosmetic substance. As societies and governments seek to improve their constituents' health and lower healthcare costs, incentives and/or mandates will be given to the consumables and cosmetic substance industry to track, maintain, and/or increase the efficacy value of consumables and cosmetic substances they handle. There will be a need, not only within each consumables and cosmetic substance industry silo to maintain or improve the efficacy value of their products, but an industry-wide solution to allow the management and tracking of evolving efficacy values across the entire cycle from creation to use. In order to manage the efficacy value of consumables and cosmetic substances across the entire cycle from creation to use, the consumables and cosmetic substance industry will need to identify, track, measure, estimate, preserve, transform, condition, and record efficacy value for consumables and cosmetic substances. Of particular importance is the measurement, estimation, and tracking of changes to the efficacy content of a consumables and cosmetic substance from creation to use. This information could be used, not only by the consumer in selecting and dispensing particular consumables and cosmetic substances, but could be used by the other consumables and cosmetic substance industry silos, including creation, preservation, transformation, and dispensing, to make decisions on how to create, handle and process consumables and cosmetic substances. Additionally, those who sell consumables and cosmetic substances to consumers could communicate perceived qualitative values of the consumables and cosmetic substance in their efforts to market and position their consumables and cosmetic substance products. Further, a determinant of price of the consumables and cosmetic substance could be particular efficacy values, and if changes to those values are perceived as desirable. For example, if a desirable efficacy value has been maintained, improved, or minimally degraded, the corresponding consumables and cosmetic substance could be marketed as a premium product. Still further, a system allowing creators, preservers, transformers, and applicators of consumables and cosmetic substances to update labeling content to reflect the most current information about the consumables and cosmetic substances would provide consumers with the information they need to make informed decisions regarding the consumables and cosmetic substances they purchase and use. Such information updates could include efficacy values of the consumables and cosmetic substance, and may further include information regarding the source, creation and other origin information for the consumables and cosmetic substance.

For example, the grower of Aloe vera generally only provides basic information such as the variety and grade of its Aloe vera leafs to an Aloe vera transformer, who extracts juices from the Aloe vera leafs, concentrates the extract, and preserves and ships the concentrate to another transformer, a skin lotion transformer, for use as an ingredient in skin lotion. The Aloe vera transformer may only tell the skin lotion transformer that the Aloe vera extract has been concentrated and frozen after extraction. The skin lotion transformer may only provide the consumer with rudimentary instructions of how to apply the skin lotion and only tell the consumer that the skin lotion contains Aloe vera extract among its ingredients. Finally, the consumer of the skin lotion will likely keep her opinions on the quality of the skin lotion to herself, unless it was an especially bad experience, where she might contact the skin lotion transformer's customer support program to complain. Very minimal, or no, information on the efficacy content of the skin lotion related to the Aloe vera extract is passed along to the consumer. The consumer knows essentially nothing about changes (generally a degradation, but could be a maintenance or even an improvement) to the efficacy content of the skin lotion related to the Aloe vera extract, particularly related to Aloe vera derived phytochemical concentrations in the skin lotion, which have occurred from creation, transformation, preservation, local storage, or dispensing. The consumer is even more unlikely to be aware of possible changes to labeling content that a creator, preserver, or transformer may just have become be aware of, such as changes in information about efficacy values of the skin lotion or changes in information regarding the source, creation and other origin information about the skin lotion or its ingredients. If communicated, such changes to labeling content could affect the purchase, local storage, dispensing, and use of the skin lotion. Further, if communicated, such changes to labeling content could affect the health, safety, and wellbeing of the consumer. It is also clear that such changes would best be communicated rapidly and by a means readily utilized by the consumer.

Consumers' needs are changing as consumers are demanding more of consumables and cosmetic substances. Consumers are also asking for more information about the consumables and cosmetic substances they consume, such as specific characteristics' relating not only to efficacy values, but to allergens, irritants, substitutions, and adulteration. For example, consumables and cosmetic substances which contain latex, dyes, ingredients derived from particular animal or plant sources, preservatives, hormones or hormones, antibiotics, etc. need to be avoided by certain consumers. However, the transformer of the skin lotion, in the prior example, has very little information to share other than possibly the source of the ingredients of the skin lotion and its processing steps in manufacturing the skin lotion. The transformer of the skin lotion does not know the efficacy value of the product after it has been locally stored and is ready for dispensing by the consumer, cannot predict changes to the efficacy value, and cannot inform a consumer of this information to enable the consumer to better meet their needs. For example, the consumer of the skin lotion may want to know what changes have occurred to efficacy values related to particular phytochemicals derived from Aloe vera, and what residual efficacy values remain, when it is purchased, during local storage in her house, and upon dispensing. Such changes in efficacy values are usually a degradation, but could be a maintenance or even improvement. There is a need to preserve, measure, estimate, store and/or transmit information regarding such efficacy values, including changes to these values, throughout the consumables and cosmetic substance supply system. Additionally, given the opportunity and a system capable of receiving and processing real time consumer feedback and updates regarding changes in the efficacy value of consumables and cosmetic substances, consumers can even play a role in updating dynamic information about the consumables and cosmetic substances they have purchased and/or are prepared to consume, such that the information is available and useful to others in the consumables and cosmetic substance supply system. Ideally, equipment and environments for local storage of consumables and cosmetic substances by consumers, such as any storage location, medicine cabinet, portable container, tray, bag, and so forth, could interact with consumables and cosmetic substance to provide such consumer feedback and updates, and preferably are utilized to meet the particular consumer's needs.

The efficacy value information for consumables and cosmetic substances provided to consumers is often minimal. When efficacy value information is provided, it is static in nature, and most likely to reflect an initial efficacy value of the corresponding consumables and cosmetic substance. There is a need to provide information about consumables and cosmetic substances in a meaningful manner. Such information needs to be presented in a manner that is responsive to the specific needs of a particular consumer. For example, consumers with a medical condition, such as diabetes, would want to track specific information regarding efficacy values associated with Insulin medicaments they purchase, locally store, and dispense, and would benefit further from knowing changes in the efficacy values or having tools to quickly indicate or estimate these changes in a retrospective, current, or prospective fashion, and even tools to report these changes, or impressions of these changes, in a real-time fashion. Consumers would want to track specific efficacy values of consumables and cosmetic substances to be aware of changes in their efficacy values, particularly a degradation in efficacy values, and for potential interactions with other consumables and cosmetic substances they are consuming or plan to consume or consumables and cosmetic substances they are consuming or plan to consume.

In fact, each silo in the consumables and cosmetic substance industry already creates and tracks some information, including efficacy value information, about their product internally. For example, the framer who grew the Aloe vera leafs knows the variety of plant, condition of the soil, the source of the water, the fertilizers and pesticides used, and can measure the leafs' efficacy content at creation. The Aloe vera extract transformer knows when it was picked, how it was transported to his processing facility, how the Aloe vera leafs were preserved, the juice extracted, concentrated, frozen, and preserved before being sent to the skin lotion transformer, when it was delivered to the skin lotion transformer, and may know what degradation to efficacy value has occurred during extraction and concentration. The skin lotion transformer likely knows the source of each ingredient of the skin lotion, how it was processed, including the processing parameters followed at his processing facility, and how it was preserved and packaged for the consumer. Not only may the skin lotion transformer know what degradation to efficacy value occurred while processing the skin lotion, it may modify its processing and post-processing preservation to minimally affect the efficacy value. Finally, a consumer generally knows how she has locally stored the skin lotion after she has purchased it, how she used the skin lotion, and whether she did or did not enjoy it.

If there was a mechanism to share this information, the quality of consumables and cosmetic substances, including efficay values, could be preserved and improved. Consumers could be better informed about consumables and cosmetic substances they select and consume, including the state, and changes in the state, of the efficacy value of the consumables and cosmetic substance throughout its lifecycle from creation to consumption. The efficiency and cost effectiveness of consumables and cosmetic substances could also be improved. Feedback within the entire chain from creator to consumer could provide a closed-loop system that could improve overall quality, efficiency value, product value and profit. For example, in the pharmaceutical supply chain, much of the product is wasted due to safety margins included in static product expiration dates. The use of more accurate tracking information, measured quality information, including historical environmental information and efficacy value information could substantially reduce such waste. Collecting, preserving, measuring and/or tracking information about a consumables and cosmetic substance in the consumables and cosmetic substance supply system, would allow needed accountability. There would be nothing to hide. Unfortunately, today there is no such system or dynamic consumables and cosmetic substance labeling.

As consumers are demanding more information about consumables and cosmetic substances they consume, they are asking for products that have higher efficacy value and would like consumables and cosmetic products to actually meet their specific requirements, particularly their needs regarding desired efficacy content of a dispensed consumables and cosmetic substance. While consumers, and all those who process, sell, and dispense consumables and cosmetic substances may obtain some information from current consumables and cosmetic substance tracking systems, such as labels, these current systems can provide only limited information.

Current packaging materials for consumables and cosmetic substances include plastics, paper, cardboard, glass, and synthetic materials. Generally, the packaging material is chosen by the manufacturer to best preserve the quality of the consumables and cosmetic substance until used by the customer. The packaging typically includes some information regarding the type of consumables and cosmetic substance, identity of the producer, country of origin, recommendations for use, expiration date, and warnings. Such packaging generally does not transmit or communicate source information of the consumables and cosmetic substance or its ingredients, such as creation information, current or historic information as to the external conditions of the packaged consumables and cosmetic substance, or current or historic information as to the internal conditions of the packaged consumables and cosmetic substance.

Consumables and cosmetic substance collectors and/or producers, such as growers (plants), ranchers (animals) or synthesizer (synthetic compounds), routinely create and collect information about their products, however, that information is generally not accessible by their customers. Even if such producers wished to provide such information to their customers, there is no current method of labeling, encoding or identifying each particular product to provide such information (even though all plants, animals and in general, consumables and cosmetic substances have a natural fingerprint). While there are limited methods and systems available, they are excessively costly, time consuming, and do not trace, or provide access to, the consumables and cosmetic substance efficacy state across the product's lifecycle. Current labels for such products include package labels, sticker labels and ink labels. These labels generally are applied to all similar products and cannot identify each particular product, only variety of products.

An important issue in the creation, preservation, transformation, dispensing, and consumption of consumables and cosmetic substances are the changes that occur in consumables and cosmetic substances due to a variety of internal and external factors. Because consumables and cosmetic substances are composed of biological, organic, and/or chemical compounds, they are generally subject to degradation. This degradation generally reduces the efficacy values of consumables and cosmetic substances. While not always true, consumables and cosmetic substances have their highest efficacy content when they are created. Currently, the consumables and cosmetic substance industry attempts to minimize the loss of efficacy values, often through the use of additives or preservatives and often through storing the consumables and cosmetic substance at specific, often narrow, storage conditions, and/or attempts to hide the loss of efficacy values from consumers. Consumers are provided with virtually no tools to help them in their attempts to determine and minimize the loss of efficacy values of the consumables and cosmetic substances they acquire, locally store, dispense, and consume.

Traditional consumables and cosmetic substance manufacturers take consumables and cosmetic substance ingredients from creators, preservers, and other transformers and transform them into consumables and cosmetic substances for use by consumers. It is understood that in some cases, consumables and cosmetic substance transformers may pass consumables and cosmetic substances they have transformed on to other consumables and cosmetic substance transformers, or to those who dispense consumables and cosmetic substances to consumers, such as to compounding pharmacies, doctor's offices, and hospitals. While these consumables and cosmetic transformers have some knowledge of the consumables and cosmetic substance ingredients they purchase, and make such selections to meet the needs of the consumers of their products, they generally do not transmit that information along to the consumers, nor change the way they transform the consumables and cosmetic substances based on the history or current condition of the consumables and cosmetic substances they receive for transformation.

Consumers of consumables and cosmetic substances are typically provided with recommendations regarding the use of consumables and cosmetic substances they have obtained. Such recommendations may include, but are not limited to: usage quantity, such as, but not limited to, dosage, volume, or weight; how to take, apply, use, or otherwise consume; frequency of use; and so forth, and are referred to herein as dispensing parameters. Current dispensing parameters are static in nature, and based on assumed efficacy values of the corresponding consumables and cosmetic substance, typically the initial efficacy values of the corresponding consumables and cosmetic substance. However, the consumer has no way of knowing the history or current condition of the consumables and cosmetic substances they have obtained, particularly as it relates to efficacy values, at the time they obtain it, during the time it is locally stored by the consumer, or at the time it is dispensed for consumption. It is understood that as used herein, consumption of consumables and cosmetic substances refers to any end use or application of the consumables and cosmetic substances by a consumer, and may include, but is not limited to, ingestion, injection, inhalation, topical application, and any other known formats for use or end application. Further, consumers have no way to change the way they locally store, dispense and consume the consumables and cosmetic substances based on the history or current condition of the consumables and cosmetic substances.

Overall, the examples herein of some prior or related systems and their associated limitations are intended to be illustrative and not exclusive. Other limitations of existing or prior systems will become apparent to those of skill in the art upon reading the following Detailed Description.

### OBJECTS OF THE INVENTION

It is an object of the present invention to preserve a consumables and cosmetic substance such that its source information and historical preservation information, including external influences on the consumables and cosmetic substance which may have caused changes in efficacy values of the consumables and cosmetic substance, herein collectively and individually referred to as ΔE, and information regarding such ΔEs or a corresponding residual efficacy value, are available to users and/or consumers of the consumables and cosmetic substance, as well as all entities of the consumables and cosmetic substance supply system, including those who create, transform, preserve, dispense, and consume consumables and cosmetic substances.

A further object of the present invention is to provide packaging which dynamically interacts with the consumables and cosmetic substance to maintain and/or improve and/or minimize degradation of the consumables and cosmetic substance being preserved, in order to maintain, improve, or minimize degradation of an efficacy value, or otherwise favorably influence a ΔE related to the consumables and cosmetic substance.

It is an object of the present invention to preserve the consumables and cosmetic substance such that its source information and/or historical preservation information, including external influences on the consumables and cosmetic substance which may have caused changes in efficacy values of the consumables and cosmetic substance, herein collectively and individually referred to as ΔE, and information regarding such ΔEs or a corresponding residual efficacy value, are available to entities outside of the consumables and cosmetic substance supply system.

In an object of the present invention, the packaging or label of a consumables and cosmetic substance tracks creation and historical information of consumables and cosmetic substance, including ΔE information as well as current information about the state of a efficacy value of the consumables and cosmetic substance.

A further object of the present invention is to provide packaging which dynamically interacts with the consumables and cosmetic substance to maintain and/or improve and/or minimize degradation of the consumables and cosmetic substance being preserved, in order to maintain, improve, or minimize degradation of an efficacy value, or otherwise favorably influence a ΔE related to the consumables and cosmetic substance, and transmits information regarding such dynamic interaction with the consumables and cosmetic substance.

It is an object of the present invention to preserve the consumables and cosmetic substance such that its source information and/or historical preservation information, including external influences on the consumables and cosmetic substance which may have caused changes in efficacy values of the consumables and cosmetic substance, herein collectively and individually referred to as ΔE, and information regarding such ΔEs or a corresponding residual efficacy value, are available by reference to a unique identifier provided with the consumables and cosmetic substance.

It is an object of the present invention to minimize and/or track ΔE of a consumables and cosmetic substance, and collect, store, and transmit the ΔE information regarding the consumables and cosmetic substance, and reference the ΔE information to a unique identifier associated with the consumables and cosmetic substance.

### SUMMARY OF THE INVENTION

In one embodiment of the present invention, the preservation system, also referred to herein as packaging, for a consumables and cosmetic substance allows for the tracking of source information, information as to the history of the consumables and cosmetic substance from the point it was packaged and/or current information on external influences on the packaged consumables and cosmetic substance which may have caused changes in efficacy values of the consumables and cosmetic substance, herein collectively and individually referred to as ΔE. It is a further embodiment of the present invention that the current information on the external influences on the packaged consumables and cosmetic substance is utilized to provide ΔE values or resulting residual efficacy values to users and/or consumers of the consumables and cosmetic substance as well as all entities of the consumables and cosmetic substance supply system, including those who create, transform, preserve, dispense, and consume consumables and cosmetic substances.

In another embodiment of the present invention the packaging or labeling for the consumables and cosmetic substance can provide information to any entity inside or outside of the consumables and cosmetic substance supply system, but preferably the consumer, related to a ΔE value or resulting residual efficacy value of the consumables and cosmetic substance.

In a further embodiment of the present invention, the packaging of the consumables and cosmetic substance can dynamically interact with the consumables and cosmetic substance to maintain, improve, or minimize degradation of an efficacy value, or otherwise favorably influence a ΔE related to the consumables and cosmetic substance.

In one embodiment of the present invention, the packaging, for a consumables and cosmetic substance allows for the tracking of source information, information as to the history of the consumables and cosmetic substance from the point it was packaged and/or current information on external influences on the packaged consumables and cosmetic substance which may have caused changes in efficacy values of the consumables and cosmetic substance, herein collectively and individually referred to as ΔE. It is a further embodiment of the present invention that the current information on the external influences on the packaged consumables and cosmetic substance is utilized to provide ΔE values or resulting residual efficacy values to entities outside of the consumables and cosmetic substance supply system.

In another embodiment of the present invention the packaging or labeling for the consumables and cosmetic substance references information related to a ΔE value or resulting residual efficacy value of the consumables and cosmetic substance by a unique identifier provided by the packaging or labeling.

In a further embodiment of the present invention, the packaging of the consumables and cosmetic substance can dynamically interact with the consumables and cosmetic substance to maintain, improve, or minimize degradation of a efficacy value, or otherwise favorably influence a ΔE related to the consumables and cosmetic substance and transmits information related to the interaction.

In one embodiment of the present invention, the packaging, for a consumables and cosmetic substance allows for the tracking of source information, information as to the history of the consumables and cosmetic substance from the point it was packaged and/or current information on external influences on the packaged consumables and cosmetic substance which may have caused changes in efficacy values of the consumables and cosmetic substance, herein collectively and individually referred to as ΔE. It is a further embodiment of the present invention that the current information on the external influences on the packaged consumables and cosmetic substance is referenced to a unique identifier provided with the packaging.

An embodiment of the present invention provides a system for the creation, collection, storage, transmission, and/or processing of information regarding dynamically labeled consumables and cosmetic substances so as to improve, maintain, or minimize degradation of efficacy value of the consumables and cosmetic substances. Additionally, the present invention provides such information for use by the creators, preservers, transformers, dispensers, and consumers of consumables and cosmetic substances. It is a preferred that this information is openly available and openly integrated at any point in time to all constituents in the consumables and cosmetic substance supply system. It is preferred that dynamic labeling provided with the consumables and cosmetic substances enables the integration and availability of the information and that this information becomes openly available and openly integrated as soon as it is created. The efficacy information creation, preservation, and transmission system of the present invention should allow the consumables and cosmetic substance supply system to improve its ability to minimize degradation of efficacy value of the consumables and cosmetic substance, and/or inform the consumer, creator, packager, transformer, or dispenser about such degradation, or ΔE. While the ultimate goal of the consumables and cosmetic substance supply system is to minimize degradation of efficacy values, or as it relates to ΔE, minimize the negative magnitude of ΔE. However, an interim goal should be providing consumers with significant information regarding any change, particularly degradation, of efficacy values of consumables and cosmetic substances, and/or component consumables and cosmetic substances thereof, consumers select and consumer, the ΔE, such that desired information regarding specific residual efficacy values can be ascertained using the ΔE. Entities within the consumables and cosmetic substance supply system that provide such ΔE information regarding consumables and cosmetic substances, particularly regarding degradation, will be able to differentiate their products from those who obscure and/or hide such information. Additionally, such entities should be able to charge a premium for products which either maintain their efficacy value, or supply more complete information about changes in their efficacy value, the ΔE.

Other advantages and features will become apparent from the following description and claims. It should be understood that the description and specific examples are intended for purposes of illustration only and not intended to limit the scope of the present disclosure.

### BRIEF DESCRIPTION OF THE DRAWINGS

The accompanying drawings, which are incorporated in and constitute a part of this specification, exemplify the embodiments of the present invention and, together with the description, serve to explain and illustrate principles of the invention. The drawings are intended to illustrate major features of the exemplary embodiments in a diagrammatic manner. The drawings are not intended to depict every feature of actual embodiments nor relative dimensions of the depicted elements, and are not drawn to scale.

FIG. 1 shows a schematic functional block diagram of a consumables and cosmetic substance supply relating to the present invention;

FIG. 2 shows a graph representing a value of a consumables and cosmetic substance which changes according to a change of condition for the consumables and cosmetic substance;

FIG. 3 shows a schematic functional block diagram of the preservation module 300 according to the present invention;

FIG. 4 shows a schematic functional block diagram of the preservation module 300 according to an alternate embodiment of the present invention;

FIG. 5 shows a schematic functional block diagram of the preservation module 300 according to an alternate embodiment of the present invention;

FIG. 6 shows a schematic functional block diagram of the preservation module 300 according to an alternate embodiment of the present invention;

FIG. 7 shows a schematic functional block diagram of the preservation module 300 according to an alternate embodiment of the present invention;

FIG. 8 shows a schematic functional block diagram of the preservation module 300 according to an alternate embodiment of the present invention;

FIG. 9 shows a schematic functional block diagram of the preservation module 300 according to an alternate embodiment of the present invention;

FIG. 10 shows a schematic functional block diagram of the preservation module 300 according to an alternate embodiment of the present invention;

FIG. 11 shows a schematic functional block diagram of the preservation module 300 according to an alternate embodiment of the present invention;

FIG. 12 shows a schematic functional block diagram of the preservation module 300 according to an alternate embodiment of the present invention; and

FIG. 13 shows a schematic functional block diagram of the preservation module 300 according to an alternate embodiment of the present invention.

In the drawings, the same reference numbers and any acronyms identify elements or acts with the same or similar structure or functionality for ease of understanding and convenience. To easily identify the discussion of any particular element or act, the most significant digit or digits in a reference number refer to the Figure number in which that element is first introduced.

### DETAILED DESCRIPTION OF THE INVENTION

Various examples of the invention will now be described. The following description provides specific details for a thorough understanding and enabling description of these examples. One skilled in the relevant art will understand, however, that the invention may be practiced without many of these details. Likewise, one skilled in the relevant art will also understand that the invention can include many other obvious features not described in detail herein. Additionally, some well-known structures or functions may not be shown or described in detail below, so as to avoid unnecessarily obscuring the relevant description.

The terminology used below is to be interpreted in its broadest reasonable manner, even though it is being used in conjunction with a detailed description of certain specific examples of the invention. Indeed, certain terms may even be emphasized below; however, any terminology intended to be interpreted in any restricted manner will be overtly and specifically defined as such in this Detailed Description section.

The following discussion provides a brief, general description of a representative environment in which the invention can be implemented. The present invention enables a consumables and cosmetic substance to interact and communicate with its preservation system in a dynamic manner through the natural changes ΔE it experiences, and further enables the preservation system to convey information associated with those changes to the consumer. As used herein, preservation systems may include, but are not limited to, any internal or external portion of a consumables and cosmetic substance package, container, carton, bottle, carton, box, bag, vessel, cup, plate, wrapper, label, or any other apparatus used to preserve, store, transfer, present, or serve a consumables and cosmetic substance.

An example of the present invention is provided of skin lotion with Aloe vera interacting, or communicating, with a portion of its container, a bottle. As the skin lotion with Aloe vera in the container ages it naturally experiences changes ΔE, particularly those efficacy values associated with its phytochemical levels, which are derived from Aloe vera. According to the present invention, a cap, a submerged coupon or indicator, or any part of the surface of the bottle can monitor one or more ΔE values and convey to a consumer the corresponding ΔE, or a corresponding current state, of the skin lotion with Aloe vera at any moment the consumer wants to know, such as when he is deciding to purchase or dispense the product.

In another example, a bottle containing Omega oil vitamin supplement could have a small area on its side with encapsulated gel in direct contact with the Omega oil vitamin supplement. As the Omega oil vitamin supplement ages, its bacteria count naturally increases, also resulting in a reduced ph. The bacteria will be able to penetrate the gel and the gel will gradually change color in response to the increasing bacteria content or concentration, indicating the increase in bacteria within the Omega oil vitamin supplement, and therefore a current state of the Omega oil vitamin supplement. For example, the gel may change from green, wherein green represents an acceptable bacteria level and associated shelf life, to yellow, wherein yellow represents a higher acceptable bacteria level and associated shorter shelf life, to red, wherein red represents the Omega oil vitamin supplement has an unacceptably high bacteria level and is not apt for consumption any more.

Alternatively, the gel may gradually change color in response to a reduction in ph, wherein changes in ph are surrogates for changes in bacteria levels. As the Omega oil vitamin supplement ages, its bacteria count naturally increases, reducing its ph. For example, the gel may change from green, wherein green represents a ph level corresponding to an acceptable bacteria level and associated shelf life, to yellow, wherein yellow represents a lower ph level and corresponding higher acceptable bacteria level and associated shorter shelf life, to red, wherein red represents a still lower ph and corresponding unacceptably high bacteria level and the Omega oil vitamin supplement is not apt for consumption any more.

It is understood that consumables and cosmetic substances, as used herein, includes, but is not limited to, synthetic compounds such as medicaments, supplements, cosmetics, hygiene, grooming, and any other substances intended for application, ingestion, injection, inhalation, introduction, or other use by a consumer, also referred to herein as consumption. The present invention may include embodiments wherein a portion of the consumables and cosmetic substance interacting or communicating with its container is segregated from a portion of the consumables and cosmetic substance to be consumed. This would be of particular benefit for packaged goods including synthetic compounds such as medicaments, in which case it would be desirable to segregate the portion of medicament interacting or communicating with the container from the portion of the medicament for consumption. In this case, the portion of the medicament interacting or communicating with the container would serve as a parallel sample of the medicament provided for consumption. This might be accomplished by providing a separate, permanently sealed cavity on or within the medicament container, its cover, its label, or any permanently sealed cavity structure known in the art, wherein the structure contains the portion of medicament intended to interact or communicate with the container. The permanently sealed cavity can interact with the portion of medicament communicating with it to convey desired ΔE information regarding the medicament. Such ΔE information may be associated with a degradation of the medicament, a residual value of the medicament, an expiration date of the medicament, or utilized in any other way to ensure the medicament's safety and efficacy when a consumer uses it.

Other examples of the present invention could include, but are not limited to, containers like jars, glasses, or cups that could detect when there is an unhealthy level of toxins, antibiotics, fungus, bacteria, pesticides, or other undesirable components in consumables and cosmetic substances intended for consumption. The principle at work is that of symbiosis, similar to that which occurs between a banana and its peel. The banana peel has a natural evolution from green to black that conveys the level of maturity of the banana. The peel reacts to the natural ΔE that occurs during the banana's maturation process, wherein the ΔEs may include changes in acidity, sugar content, and bacteria level. The ΔEs of the banana independently and collectively have an effect on the aesthetic values of the banana peel, which in turn conveys to the consumer when and how the banana may best be consumed. For example, a green peel indicates that the banana is not yet ripe and should not be eaten. Yellow indicates that it may be suitable for consumption, but will not be very sweet. Yellow with a few black spots indicates that it is suitable for consumption, and will be sweat. Mostly black indicates that it is suitable for use in baked goods or to be fried. Very black indicates that it is no longer suitable for consumption. In this same manner when the peel has been punctured or torn and the maturating process is accelerated as more oxygen than normal contacts the banana, the banana peel quickly turns black alerting the consumer. Therefore the consumer does not have to rely on a static expiration date to determine the banana's suitability for consumption.

Although not required, aspects of the invention may be described below in the general context of computer-executable instructions, such as routines executed by a general-purpose data processing device (e.g., a server computer or a personal computer). Those skilled in the relevant art will appreciate that the invention can be practiced with other communications, data processing, or computer system configurations, including: wireless devices, Internet appliances, hand-held devices (including personal digital assistants (PDAs)), wearable computers, all manner of cellular or mobile phones, multi-processor systems, microprocessor-based or programmable consumer electronics, set-top boxes, network PCs, mini-computers, mainframe computers, and the like. Indeed, the terms “controller,” “computer,” “server,” and the like are used interchangeably herein, and may refer to any of the above devices and systems.

While aspects of the invention, such as certain functions, are described as being performed exclusively on a single device, the invention can also be practiced in distributed environments where functions or modules are shared among disparate processing devices. The disparate processing devices are linked through a communications network, such as a Local Area Network (LAN), Wide Area Network (WAN), or the Internet. In a distributed computing environment, program modules may be located in both local and remote memory storage devices.

Aspects of the invention may be stored or distributed on tangible computer-readable media, including magnetically or optically readable computer discs, hard-wired or preprogrammed chips (e.g., EEPROM semiconductor chips), nanotechnology memory, biological memory, or other data storage media. Alternatively, computer implemented instructions, data structures, screen displays, and other data related to the invention may be distributed over the Internet or over other networks (including wireless networks), on a propagated signal on a propagation medium (e.g., an electromagnetic wave(s), a sound wave, etc.) over a period of time. In some implementations, the data may be provided on any analog or digital network (packet switched, circuit switched, or other scheme).

In some instances, the interconnection between modules is the internet, allowing the modules (with, for example, WiFi capability) to access web content offered through various web servers. The network may be any type of cellular, IP-based or converged telecommunications network, including but not limited to Global System for Mobile Communications (GSM), Time Division Multiple Access (TDMA), Code Division Multiple Access (CDMA), Orthogonal Frequency Division Multiple Access (OFDM), General Packet Radio Service (GPRS), Enhanced Data GSM Environment (EDGE), Advanced Mobile Phone System (AMPS), Worldwide Interoperability for Microwave Access (WiMAX), Universal Mobile Telecommunications System (UMTS), Evolution-Data Optimized (EVDO), Long Term Evolution (LTE), Ultra Mobile Broadband (UMB), Voice over Internet Protocol (VoIP), Unlicensed Mobile Access (UMA), etc.

The modules in the systems can be understood to be integrated in some instances and in particular embodiments, only particular modules may be interconnected.

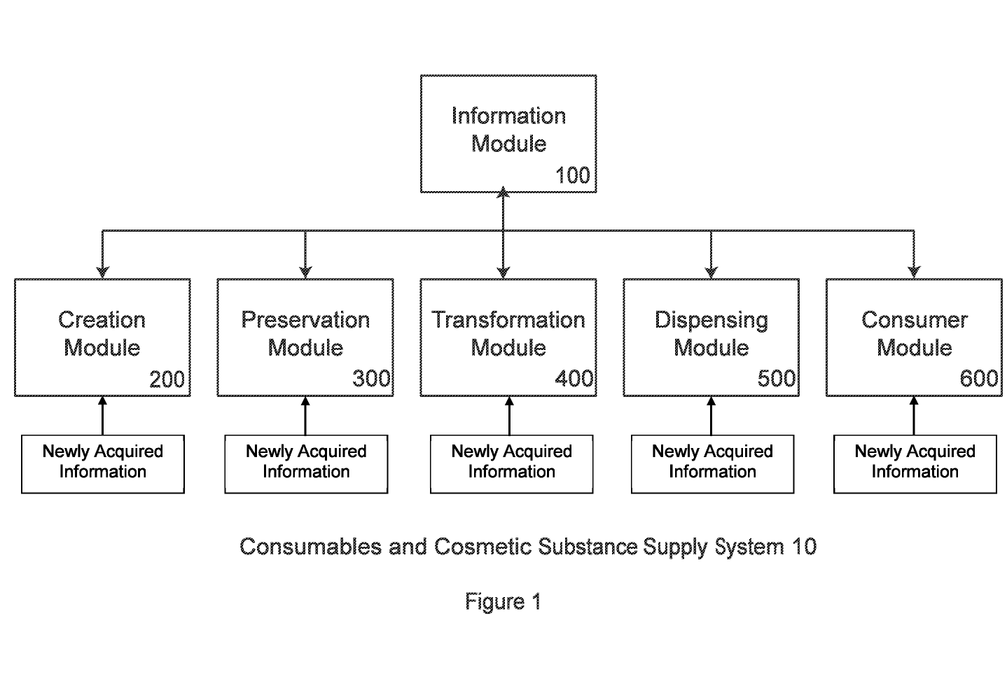

FIG. 1 shows the components of a consumables and cosmetic substance industry 10. It should be understood that this could be the consumables and cosmetic substance ecosystem for human consumption, but could also be the consumables and cosmetic substance industry for animal consumption, such as the veterinary medicine and animal grooming industries. A goal of the present invention for the consumables and cosmetic substance industry 10 is to create, preserve, transform and trace the change in efficacy values of consumables and cosmetic substances, collectively and individually also referred to herein as ΔE, through their creation, preservation, transformation, local storage, dispensing, and consumption. While the consumables and cosmetic substance industry 10 can be composed of many companies or businesses, it can also be integrated into combinations of business serving many roles, or can be one business or even individual. Since ΔE is a measure of the change in an efficacy value of a corresponding consumables and cosmetic substance, knowledge of a prior efficacy value (also referred to as prior efficacy state) of a consumables and cosmetic substance and the ΔE value will provide knowledge of the residual efficacy value (also referred to as current efficacy value or current efficacy state).

Module 200 is the creation module. This can be a system, organization, or individual which creates and/or originates consumables and cosmetic substances. Examples of this module include, but are not limited to, a farm that grows Aloe vera from which cosmetic products are made; a ranch that raises pigs from which porcine derived Insulin medicaments are made; an aquaculture farm that grows salmon from which Omega oil supplements are derived; a factory that synthesizes chemical compounds; a collector of wild Gen sing root; and so forth.

Preservation module 300 is a preservation system for storing, preserving and protecting the consumables and cosmetic substances created by creation module 200, or transformed by the transformation module 400. Once the consumables and cosmetic substance has been created or transformed, generally, it will need to be packaged in some manner for its transition to other modules in the consumables and cosmetic substances industry 10. While preservation module 300 is shown in a particular position in the consumables and cosmetic substance industry 10, following the creation module 200, it should be understood that the preservation module 300 actually can be placed anywhere consumables and cosmetic substances need to be stored and preserved during their transition from creation to consumption. It is understood that a consumables and cosmetic substance may experience more than one preservation event, and that such preservation events may even be considered to include the local storage of the consumables and cosmetic substance, such as in a local storage environment, a local storage container, or a dispenser prior to consumption.

Transformation module 400 is a consumables and cosmetic substance processing system, such as a manufacturer who processes raw materials such as raw vitamin compounds and carriers into multi vitamin tablets. Transformation module 400 could also be an Aloe vera extract concentrate manufacturer who receives raw components, or ingredients, also referred to herein as component consumables and cosmetic substances, from preservation module 300 (for example Aloe vera leafs in a sealed, temperature controlled container) and processes them into an Aloe vera extract concentrate. While transformation module 400 is depicted as one module, it will be understood that consumables and cosmetic substances may be transformed by a number of transformation modules 400 on their path to consumption.

Dispensing module 500 is a module for dispensing consumables and cosmetic substances immediately before consumption. Dispensing module 500 may comprise, but is not limited to, a volumetric-based dispensing system, a weight-based dispensing machine, a counting device, a controlled storage environment, a storage container tracking storage conditions such as temperature, an individual such as a doctor, pharmacist, nurse, patient, etc. It may also be systems used by commercial establishments to prepare consumables and cosmetic substance for consumers, such as equipment used by a hospital or a compounding pharmacy, or other devices located at businesses which provide consumables and cosmetic substances to consumers. Such consumables and cosmetic substances could be for consumption at the business or for the consumer to take out from the business. Dispensing module 500 can also be any combination of these systems, machines, devices, equipment, or individuals used to dispense consumables and cosmetic substances for consumption by consumers.

Consumer module 600 collects information from the living entity which consumes the consumables and cosmetic substance which has passed through the various modules from creation to consumption. The consumer can be a human being, but could also be an animal, such as pets, zoo animals and livestock, which may themselves comprise consumables and cosmetic substances or nutritional substances for other consumption chains. Consumers could also be plant life which consumes consumables and cosmetic substances to grow, such as plants that are provided with chemical fertilizers or insecticides.

Information module 100 receives and transmits information regarding a consumables and cosmetic substance between each of the modules in the consumables and cosmetic substance industry 10 including, the creation module 200, the preservation module 300, the transformation module 400, the dispensing module 500, and the consumer module 600. The consumables and cosmetic substance information module 100 can be an interconnecting information transmission system which allows the transmission of information between various modules. Information module 100 contains a database, also referred to herein as a dynamic efficacy value database, where information regarding the consumables and cosmetic substance resides, particularly ΔE information for the consumables and cosmetic substance. Information module 100 may also contain a massive database of sensed physical attribute values for known consumables and cosmetic substances at known efficacy states, also referred to herein as a consumables and cosmetic substance attribute library, which can be utilized for determining the identity and current efficacy state of a consumables and cosmetic substance. Information module 100 can be connected to the other modules by a variety of communication systems, such as paper, computer networks, the internet and telecommunication systems, such as wireless telecommunication systems. In a system capable of receiving and processing real time consumer feedback and updates regarding changes in an efficacy value of a corresponding consumables and cosmetic substance, or ΔE, consumers can even play a role in updating the dynamic efficacy value database with observed or measured information about the consumables and cosmetic substances they have purchased and/or dispensed for consumption and/or consumed, so that the information is available and useful to determine a corresponding ΔE, and may further be available to others in the consumables and cosmetic substance supply system.

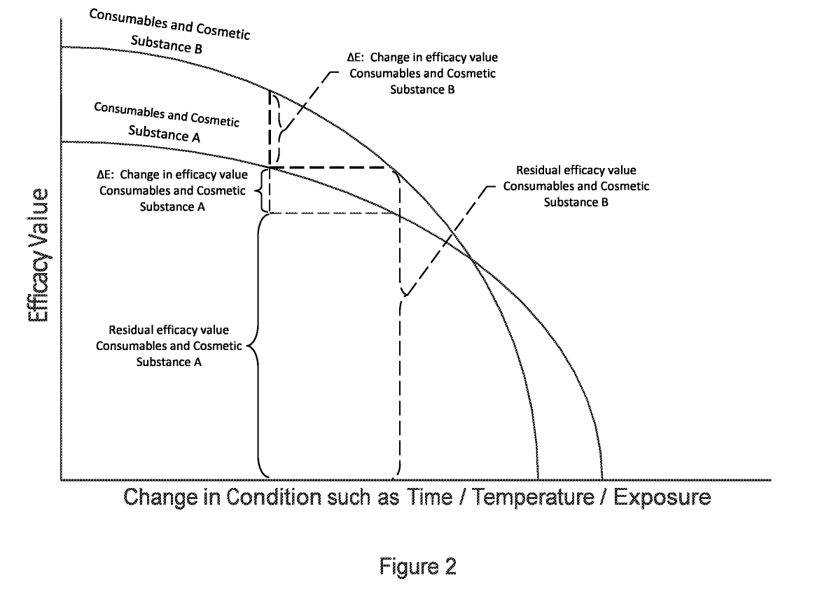

FIG. 2 is a graph showing the function of how an efficacy value of a consumables and cosmetic substance varies over the change in a condition of the consumables and cosmetic substance. Plotted on the vertical axis of this graph can be the efficacy value of a corresponding consumables and cosmetic substance. Plotted on the horizontal axis can be the change in condition of the consumables and cosmetic substance over a variable such as time, temperature, location, and/or exposure to environmental conditions. This exposure to environmental conditions can include: exposure to air, including the air pressure and partial pressures of oxygen, carbon dioxide, water, or ozone; airborne chemicals, pollutants, allergens, dust, smoke, carcinogens, radioactive isotopes, or combustion byproducts; exposure to moisture; exposure to energy such as mechanical impact, mechanical vibration, irradiation, heat, or sunlight; or exposure to materials such as packaging. The function plotted as consumables and cosmetic substance A could show a ΔE for skin lotion with Aloe vera, such as the degradation of an Aloe vera based phytochemical efficacy value over time. Any point on this curve can be compared to another point to measure and/or describe the change in efficacy value, or the ΔE, of consumables and cosmetic substance A. The function plotted as consumables and cosmetic substance B, also skin lotion with Aloe vera, shows the degradation in the same efficacy value, or the ΔE, of an Aloe vera based phytochemical efficacy value over time. Consumables and cosmetic substance B starts out with a higher efficacy value than consumables and cosmetic substance A, but degrades over time more quickly than consumables and cosmetic substance A.

In this example, where consumables and cosmetic substance A and consumables and cosmetic substance B are skin lotion with Aloe vera, this ΔE information regarding the degradation profile of efficacy value for each skin lotion could be used by the consumer in the selection and/or consumption of the corresponding skin lotion. If the consumer has this information at time zero when selecting a skin lotion product for purchase, the consumer could consider when she plans to consume the skin lotion and whether that is over a short time period or a long time period. For example, if the consumer planned to consume the skin lotion prior to the point when the curve represented by consumables and cosmetic substance B crosses the curve represented by consumables and cosmetic substance A, then the consumer should choose the skin lotion represented by consumables and cosmetic substance B because it has a higher efficacy value until it crosses the curve represented by consumables and cosmetic substance A. However, if the consumer expects to consume at least some of the skin lotion at a point in time after the time when the curve represented by consumables and cosmetic substance B crosses the curve represented by consumables and cosmetic substance A, then the consumer might choose to select the skin lotion represented by consumables and cosmetic substance A, even though the skin lotion represented by consumables and cosmetic substance A has a lower efficacy value than the skin lotion represented by consumables and cosmetic substance B at an earlier time. This change to a desired efficacy value in a consumables and cosmetic substance over a change in a condition of the consumables and cosmetic substance described in FIG. 2 can be measured and/or controlled throughout the consumables and cosmetic substance supply system 10. This example demonstrates how dynamically generated information regarding a ΔE of a consumables and cosmetic substance, in this case a change in efficacy value of skin lotion with Aloe vera, can be used to understand a rate at which that efficacy value changes or degrades; when that efficacy value expires; and a residual efficacy value of the consumables and cosmetic substance over a change in a condition of the consumables and cosmetic substance, in this example a change in time. This ΔE information could further be used to determine a best consumption date for consumables and cosmetic substance A and B, which could be different from each other depending upon the dynamically generated information generated for each. Still further, this ΔE information can be used to adaptively dispense the skin lotion such that the dispensed efficacy content meets the consumer's needs.

In FIG. 1, Creation module 200 can dynamically encode consumables and cosmetic substances to enable the tracking of changes in efficacy value of the consumables and cosmetic substance, or ΔE. This dynamic encoding, also referred to herein as a dynamic information identifier, can replace and/or complement existing consumables and cosmetic substance marking systems such as barcodes, labels, and/or ink markings. This dynamic encoding, or dynamic information identifier, can be used to make consumables and cosmetic substance information from creation module 200 available to information module 100 for use by preservation module 300, transformation module 400, dispensing module 500, and/or consumption module 600, which includes the ultimate consumer of the consumables and cosmetic substance. A key resource also available through module 100 is formulation information regarding substances that may utilize the consumables and cosmetic substances as components. The ΔE information combined with formulation information from module 100 will not only be of great benefit to the consumer in understanding and accomplishing the efficacy values desired, it will even help dispel misunderstandings that consumers may have about particular efficacy values of consumables and cosmetic substances or the combination or consumables and cosmetic substances. One method of marking the consumables and cosmetic substance with a dynamic information identifier by creation module 200, or any other module in consumables and cosmetic substance supply system 10, could include an electronic tagging system, such as the tagging system manufactured by Kovio of San Jose, Calif., USA. Such thin film chips can be used not only for tracking consumables and cosmetic substances, but can include components to measure attributes of consumables and cosmetic substances, and record and transmit such information. Such information may be readable by a reader including a satellite-based system. Such a satellite-based consumables and cosmetic substance information tracking system could comprise a network of satellites with coverage of some or all the surface of the earth, so as to allow the dynamic efficacy value database of information module 100 real time, or near real time updates about a ΔE of a particular consumables and cosmetic substance. In turn, this information is openly available and openly integrated at any point in time to all constituents in the consumables and cosmetic substance supply system. It is also preferred that this information becomes openly available and openly integrated as soon as it becomes available.

Preservation module 300 includes packers and shippers of consumables and cosmetic substances. The tracking of changes in efficacy values, or ΔE, during the preservation period within preservation module 300 allows for dynamic expiration dates for consumables and cosmetic substances. For example, expiration dates for medicament products are currently based generally only on time using assumptions regarding minimal conditions at which the corresponding medicament products are maintained. This extrapolated expiration date is based on a worst-case scenario for when the product becomes unsafe to consume during the preservation period, or when an efficacy value drops below an acceptable minimum value. In reality, the degradation of medicament products may be significantly less than this worst-case. If preservation module 300 could measure or derive the actual degradation information such as ΔE, an actual expiration date, referred to herein as a dynamic expiration date, can be determined dynamically, and could be significantly later in time than an extrapolated expiration date. This would allow the consumables and cosmetic substance supply system to dispose of fewer products due to expiration dates. This ability to dynamically generate expiration dates for consumables and cosmetic substances is of particular significance when consumables and cosmetic substances contain few or no preservatives, which is often the case.

It should be noted that a dynamic expiration date need not be indicated numerically (i.e., as a numerical date) but could be indicated symbolically as by the use of colors—such as green, yellow and red employed on semaphores—or other designations. In those instances, the dynamic expiration date would not be interpreted literally but, rather, as a dynamically-determined advisory date. In practice a dynamic expiration date will be provided for at least one component of a single or multi-component consumables and cosmetic substance. For multi-component consumables and cosmetic substances, the dynamic expiration date could be interpreted as a “best” date for consumption for particular components. Consumers of consumables and cosmetic substances provided with dynamic labeling comprising dynamic information identifiers could readily access this type of information regarding dynamic expiration dates for the consumables and cosmetic substances.

By law, in many localities, consumables and cosmetic substance processors such as those in transformation module 400 are required to provide consumables and cosmetic substance information regarding their products. Often, this information takes the form of an ingredient table applied to the packaging of the consumables and cosmetic substance. Currently, the information in this ingredient table is based on averages or minimums for their typical product. Using the consumables and cosmetic substance information from information module 100 provided by creation module 200, preservation module 300, and/or information from the transformation of the consumables and cosmetic substance by transformation module 400, and consumer feedback and updates related to ΔE, preferably obtained through or provided by local storage environments, local storage containers, and local storage coupons, the consumables and cosmetic substance processor could include a dynamically generated efficacy value table, also referred to herein as a dynamic efficacy value table, for the actual consumables and cosmetic substance being supplied to consumers and further being locally stored by consumers. The information in such a dynamic efficacy value table could be used by dispensing module 500 for adaptively dispensing the consumables and cosmetic substance, and/or used by consumption module 600, so as to allow the ultimate consumer the ability to select the most desirable consumables and cosmetic substance which meets their needs, and/or to track information regarding consumables and cosmetic substances consumed.

Information about changes in efficacy values of consumables and cosmetic substances, or ΔE, is particularly useful in the dispensing module 500, as it allows knowing, or estimating, a ΔE prior to dispensing, and the corresponding pre-dispensing state of the efficacy values of the consumables and cosmetic substance, including the changes in efficacy values occurring during local storage of the consumables and cosmetic substance, and further enables the determination of dispensing parameters responsive to the ΔE occurring prior to dispensing. The dispensing module 500 can thereby provide adaptive dispensing parameters, such as by modifying existing or baseline dispensing parameters, to deliver a desired amount of efficacy content. The pre-dispensing ΔE and corresponding efficacy value of a consumables and cosmetic substance is not tracked or provided to the consumer by existing local storage environments, local storage containers, dispensing devices or individuals. However, using information provided by information module 100 from creation module 200, preservation module 300, transformation module 400, and consumer feedback and updates related to ΔE, preferably obtained through or provided by local storage environments, local storage containers, and local storage coupons, and/or information measured or generated by dispensing module 500, and/or consumer input regarding efficacy value or a desired amount of efficacy content provided through the dispensing module 500, dispensing module 500 can provide the consumer with the actual, and/or estimated change in efficacy values of the consumables and cosmetic substance, or ΔE, prior to dispensing, and can further provide adaptive dispensing parameters responsive to the ΔE and the consumer's input to deliver a desired amount of efficacy content.

The information regarding consumables and cosmetic substances provided by information module 100 to consumption module 600 can replace or complement existing traditional information sources such as, but not limited to, traditional labeling, consumables and cosmetic substance websites like www.webmd.com, www.doctoroz.com, and consumables and cosmetic substance manufacturer's websites. Through the use of specific information regarding a consumables and cosmetic substance from information module 100, consumers can use consumption module 600 to select consumables and cosmetic substances according to ΔE information and residual efficacy values. This will further allow consumers to make informed decisions regarding consumables and cosmetic substance additives, preservatives, origins, traceability, and other consumables and cosmetic substance attributes that may also be tracked through the information module 100. This information can be provided by consumption module 600 through personal computers, laptop computers, tablet computers, and/or smartphones. Software running on these devices can include dedicated computer programs, modules within general programs, and/or smartphone apps. An example of an analogous smartphone app for consumables and cosmetic substances is the iOS ShopNoGMO from the Institute for Responsible Technology. This iPhone app allows consumers access to information regarding non-genetically modified organisms they may select. Additionally, consumption module 600 may provide information for the consumer to operate dispensing module 500 with adaptive dispensing parameters, wherein the adaptive dispensing parameters are responsive to a ΔE or corresponding residual efficacy value of the consumables and cosmetic substance being dispensed and may further be responsive to the consumer's input related to the ΔE, the corresponding efficacy value, or the corresponding amount of efficacy content to be dispensed. In this way, the amount of efficacy content in the dispensed consumables and cosmetic substance can be optimized or maintained, according to, but not limited to: a target amount based on the consumer's input regarding his needs or preference; a target amount established by the provider of the consumables and cosmetic substance, such as the transformer; a target amount established by a dispenser of the consumables and cosmetic substance, such as a service provider; or a target amount equal to a predetermined amount of efficacy content recommended by the labeling or product information provided with the consumables and cosmetic substance.

Through the use of consumables and cosmetic substance information available from information module 100, the consumables and cosmetic substance supply system 10 can track efficacy values of consumables and cosmetic substances. Using this information, consumables and cosmetic substances travelling through consumables and cosmetic substance supply system 10 can be dynamically valued and priced according to efficacy values. For example, consumables and cosmetic substances with longer dynamic expiration dates (longer shelf life) may be more highly valued than consumables and cosmetic substances with shorter expiration dates. Additionally, consumables and cosmetic substances with higher efficacy values may be more highly valued, not just by the consumer, but also by each entity within consumables and cosmetic substance supply system 10. This is because each entity will want to start with a consumables and cosmetic substance with higher efficacy value before it performs its function and passes the consumables and cosmetic substance along to the next entity. Therefore, both the starting efficacy values and the ΔE associated with those values are important factors in determining or estimating an actual, or residual, efficacy value of a consumables and cosmetic substance, and accordingly are important factors in establishing dynamically valued and priced consumables and cosmetic substances.

During the period of implementation of the present inventions, there will be consumables and cosmetic substances being marketed including those benefiting from the tracking of dynamic efficacy information such as ΔE, also referred to herein as information-enabled consumables and cosmetic substances, and consumables and cosmetic substances which do not benefit from the tracking of dynamic nutritional information such as ΔE, which are not information enabled and are referred to herein as dumb consumables and cosmetic substances. Information-enabled consumables and cosmetic substances would be available in virtual internet marketplaces, as well as traditional marketplaces. Because of information provided by information-enabled consumables and cosmetic substances, entities within the consumables and cosmetic substance supply system 10, including consumers, would be able to review and select information-enabled consumables and cosmetic substances for purchase. It should be expected that, initially, the information-enabled consumables and cosmetic substances would enjoy a higher market value and price than dumb consumables and cosmetic substances. However, as information-enabled consumables and cosmetic substances become more the norm, the cost savings from less waste due to degradation of information-enabled consumables and cosmetic substances could lead to their price actually becoming less than dumb consumables and cosmetic substances.

For example, the manufacturer of a skin lotion with Aloe vera would prefer to use Aloe vera concentrate of a high efficacy value in the production of its product, the skin lotion with Aloe vera, so as to produce a premium product of high efficacy value. Depending upon the levels of the efficacy values in the skin lotion with Aloe vera, the manufacturer may be able to charge a premium price and/or differentiate its product from that of other manufacturers. When selecting the Aloe vera concentrate to be used in the ready-to-eat dinner, the manufacturer will seek Aloe vera concentrate of high efficacy value from preservation module 300 that meets its requirements for efficacy value. The packager/shipper of preservation module 300 would also be able to charge a premium for Aloe vera concentrate which has high efficacy values upon delivery to the manufacturer of the skin lotion, and therefore is incentivized to select Aloe vera concentrate of high efficacy value from the transformation module 400, such as a processer of Aloe vera concentrate. The concentrate processer will be able to charge a premium for Aloe vera concentrate of high efficacy value, and will select Aloe vera leafs of high efficacy value from the preservation module 300, such as a produce packager/shipper. The produce packager/shipper of preservation module 300 would also be able to charge a premium for Aloe vera leafs which have high efficacy values, and therefore is inclined to select Aloe vera leafs of high efficacy value from the grower of creation module 200, who will also be able to charge a premium for Aloe vera leafs of high efficacy value.

Further, the consumer of the skin lotion with Aloe vera may want to, or in the case of a hotel, spa, hospital, or any other regulated service provider or dispenser, may be required to, track the efficacy value of the skin lotion during its local storage. Local storage environments and local storage containers providing the benefits disclosed herein enable such tracking by making information related to ΔE during local storage available to information module 100 for updating the dynamic efficacy values of consumables and cosmetic substances.

The change in efficacy value for a consumables and cosmetic substance, or ΔE, tracked through the consumables and cosmetic substance supply system 10 by consumables and cosmetic substance information from information module 100 can be preferably determined from measured information. However, some or all such consumables and cosmetic substance ΔE information may be derived through measurements of environmental conditions of the consumables and cosmetic substance as it travels through the consumables and cosmetic substance supply system 10. Additionally, some or all of the consumables and cosmetic substance ΔE information can be derived from ΔE data of other consumables and cosmetic substances which have travelled through consumables and cosmetic substance supply system 10. Consumables and cosmetic substance ΔE information can also be derived from laboratory experiments performed on other consumables and cosmetic substances, which may approximate conditions and/or processes to which the actual consumables and cosmetic substance has been exposed. Further, consumer feedback and updates regarding observed or measured changes in the efficacy value of information-enabled consumables and cosmetic substances can play a role in updating ΔE information. Also, a creator, preserver, transformer, or dispenser may revise ΔE information, or information regarding other attributes of information-enabled consumables and cosmetic substances they have previously created or processed, based upon newly acquired information affecting the ΔE or the other attributes.

For example, laboratory experiments can be performed on over-the-counter medicaments to determine the effect on, or change in, corresponding efficacy values, for a variety of environmental conditions the over-the-counter medicaments may be exposed to during packaging and shipment in preservation module 300, or during local storage by a consumer or dispenser. Using this experimental data, tables and/or algorithms could be developed which would predict the level of change of efficacy values, or ΔE, for a particular over-the-counter medicament based upon information collected regarding the environmental conditions to which the over-the-counter medicament was exposed during its time in preservation module 300 or local storage. While the ultimate goal for consumables and cosmetic substance supply system 10 would be the actual measurement of efficacy values to determine ΔE, use of derived efficacy values from experimental data to determine ΔE would allow improved logistics planning because it provides the ability to prospectively estimate changes to efficacy values, or ΔE, and because it allows more accurate tracking of changes to efficacy values, or ΔE, while technology and systems are put in place to allow actual measurement.

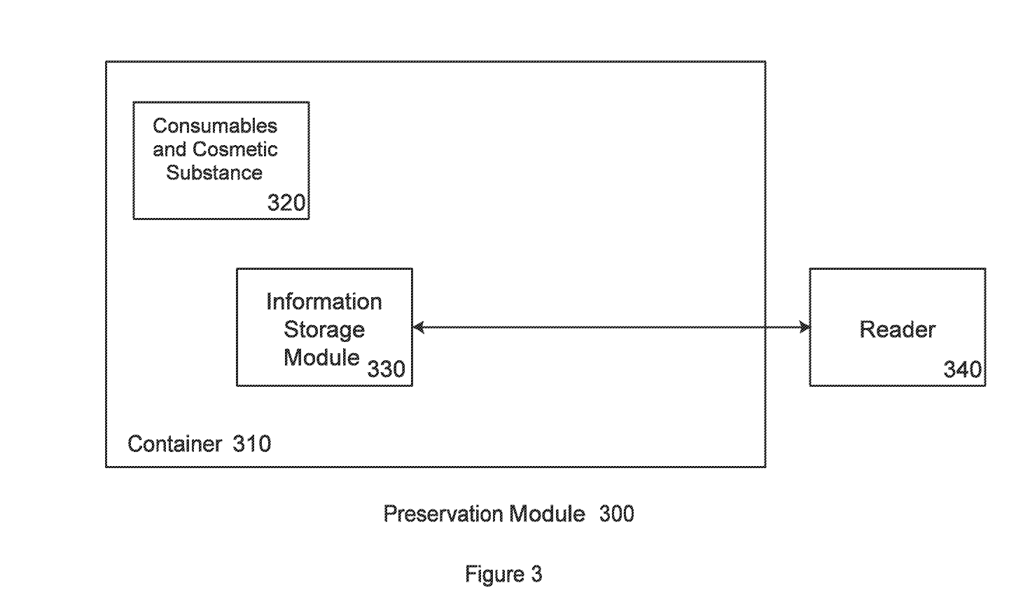

FIG. 3 shows an embodiment of the preservation module of the present invention. Preservation system 300 includes a container 310 which contains consumables and cosmetic substance 320. Also included in container 310 is information storage module 330 which can be connected to an external reader 340. In this embodiment, information storage module 330 contains information regarding the consumables and cosmetic substance 320. This information can include creation information from the creation of the consumables and cosmetic substance 320. However, information in the information storage module 320 might include identification information, information regarding prior preservation and transformation of the consumables and cosmetic substance 320, information related to ΔE, and other historic information. A shipper, or user, of container 310 can operatively connect to information storage module 330 using reader 340 to retrieve information stored therein. It is understood that reader 340 may connect to information storage module 330 by any connection methodologies known to one skilled in the art, including, but not limited to, physical connection, electronic connection, optical connection, wireless connection, or other near-field technologies. Such methodologies could be accomplished by local or remote interrogation of information storage module 330 by reader 340, or may be accomplished by transmission, such as by information storage module 330 transmitting data to a remote or local reader 340.

In an alternate embodiment reader 340 can also write to information storage module 330. In this embodiment, information regarding the container and/or consumables and cosmetic substance 320 can be modified or added to information storage module 330 by the user or shipper.

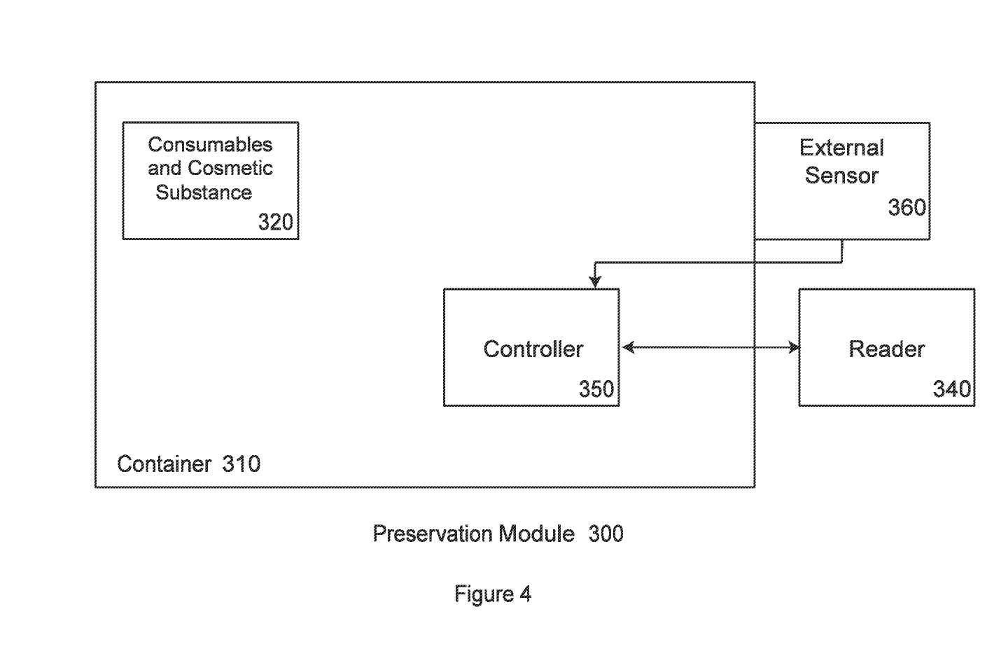

FIG. 4 shows another embodiment of preservation system 300 wherein container 310 contains consumables and cosmetic substance 320 as well as controller 350. Controller 350 is connected to external sensor 360 located either inside, on the surface of, or external to container 310 such that external sensor 360 can obtain information regarding the environment external to container 310. Controller 350 and exterior sensor 360 can take the form of electronic components such as a micro-controller and an electronic sensor, or any sensor known to one skilled in the art. For example, the controller-sensor combination may also be chemical or organic materials which perform the same function, such as a liquid crystal sensor/display.

When the shipper or user of container 310 desires information from external sensor 360 the shipper or user can use reader 340 to query the controller 350 as to the state of external sensor 360. In the electronic component embodiment, reader 340 could be a user interface device such as a computer which can be electronically connected to controller 350. In the liquid crystal sensor/display, the ready could be a human looking at the display.

In one embodiment, reader 340 can be directly connected to external sensor 360 to obtain the information from external sensor 360 without need of a controller 350. In another embodiment, external sensor 360 provides information to controller 350 which is presented as a visual display to the shipper or user. Finally, external sensor 360 could provide information directly to the user or shipper by visual means such as a temperature sensitive liquid crystal thermometer.

In an additional embodiment, controller 350 can modify the operation of container 310 so as modify the preservation capabilities of container 310, so as to favorably influence a ΔE of the consumables and cosmetic substance. For example, if the exterior environment of container 310 would adversely affect the consumables and cosmetic substance 320, for example to cause an undesirable negative ΔE, container 310 could adjust the internal environment of container 310 responsive to the information from sensor 360, to better preserve the consumables and cosmetic substance. If consumables and cosmetic substance needs to be kept within a certain temperature range to preserve its efficacy properties, and the external sensor 360 provide exterior temperature information to controller 350, controller 350 could adaptively modify container 310 so as to maintain consumables and cosmetic substance 320 within the required temperature range.

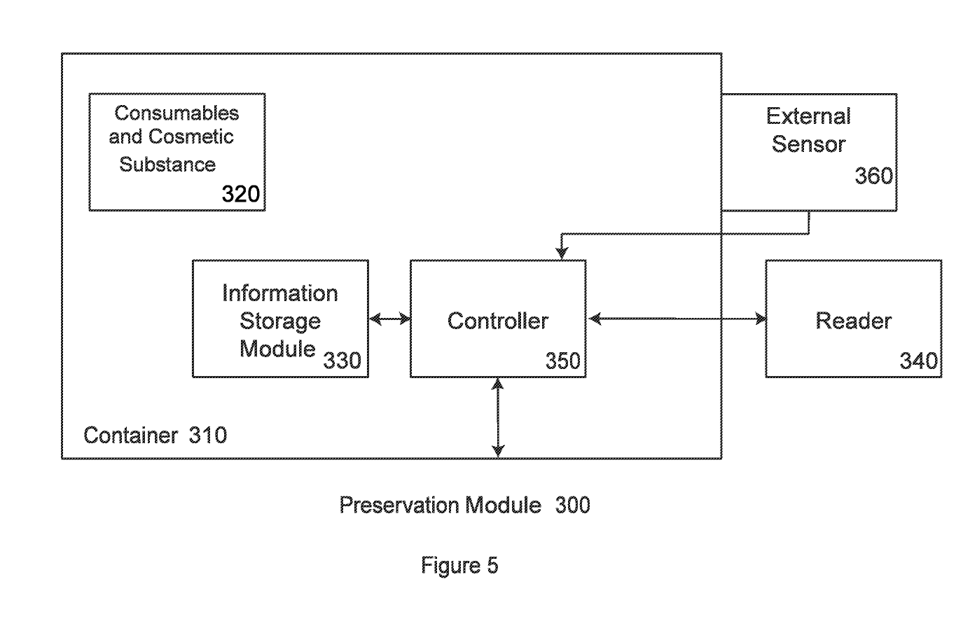

In FIG. 5, preservation system 300 includes container 310 which contains consumables and cosmetic substance 320, controller 350, and information storage module 330. External sensor 360 is positioned such that it can provide information on the exterior environment to container 310. Information from the external sensor and information storage module can be retrieved by connecting reader 340 to container 310.

In this embodiment, information regarding the external environment sensed by external sensor 360 and provided to controller 350 can be stored in information storage module 330. This storage of external environment can be used to record a history the external environment container 310 has been subjected to. This would allow the shipper or user of container 310 to understand the external environment the container has been subjected to during the time it has preserved the consumables and cosmetic substance. Such information can be used to determine any number of ΔE values for the consumables and cosmetic substance and if the consumables and cosmetic substance is no longer safe for consumption or has been degraded such that one or more ΔE values of the consumables and cosmetic substance is no longer in an optimal state or has expired. Additionally, the user of the consumables and cosmetic substance could modify its transformation, dispensing, or consumption according to any changes, or ΔEs that may have occurred because of the external conditions of the container.

Additionally, in this embodiment, information storage module 340 could contain other information regarding the consumables and cosmetic substance, including creation information, identification information, and/or prior preservation and transformation information.

In an additional embodiment, controller 350 can modify the operation of container 310 so as modify the preservation capabilities of container 310, so as to favorably influence a ΔE of the consumables and cosmetic substance. For example, if the exterior environment of container 310 would adversely affect the consumables and cosmetic substance 320, container 310 could adjust the internal environment of container 310 responsive to the information regarding the exterior environment to better preserve the consumables and cosmetic substance. Controller 350 can analyze the historic information from external sensor 360, stored in information storage module 330 to determine any long-term exterior environmental conditions. If consumables and cosmetic substance needs to be kept within a certain temperature range to preserve its efficacy properties, and the external sensor 360 provide exterior temperature information to controller 350, controller 350 could adaptively modify container 310 responsive to the exterior temperature so as to maintain consumables and cosmetic substance 320 within the required temperature range, thus minimizing degradation of one or more efficacy values of the consumables and cosmetic substance 320.

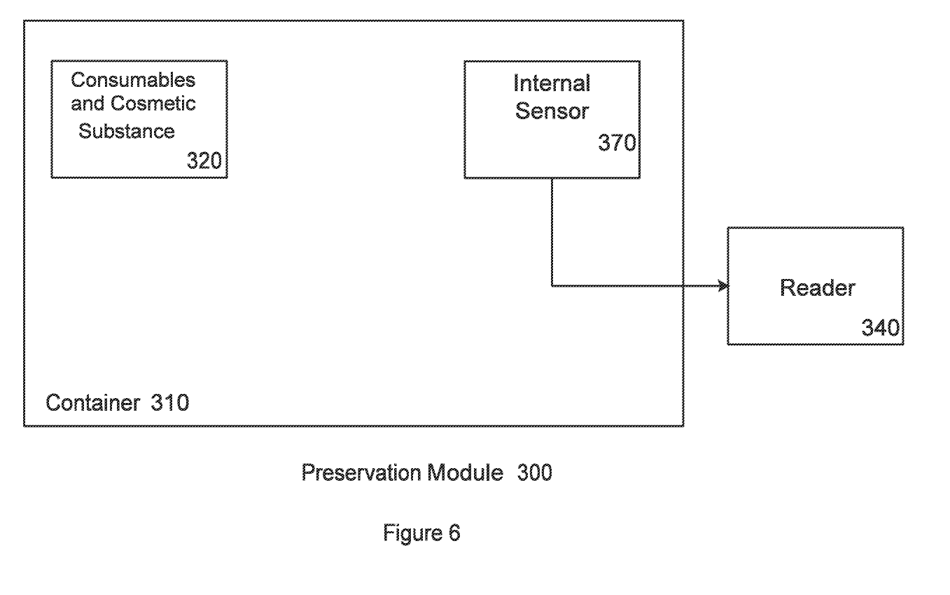

FIG. 6 shows an embodiment of preservation system 300 wherein container 310 contains consumables and cosmetic substance 320 as well as internal sensor 370 located either inside, or on the surface of, container 310, such that internal sensor 370 can obtain information regarding the environment internal to container 310. Internal sensor 370 can be connected in any known fashion to reader 340 to obtain the interior conditions of container 310. Internal sensor 370 and reader 340 can take the form of electronic components such as an electronic sensor and electronic display, or any other known sensor/reader formats. For example, the reader-sensor combination may be chemical or organic materials which perform the same function, such as a liquid crystal sensor/display. It is understood that the connection between internal sensor 370 and reader 340 may be by any connection methodologies known to one skilled in the art, including, but not limited to, physical connection, electronic connection, optical connection, wireless connection, or other near-field technologies. Such methodologies could be accomplished by local or remote interrogation of information sensor 370 by reader 340, or may be accomplished by transmission, such as by sensor 370 transmitting data to a remote or local reader 340.

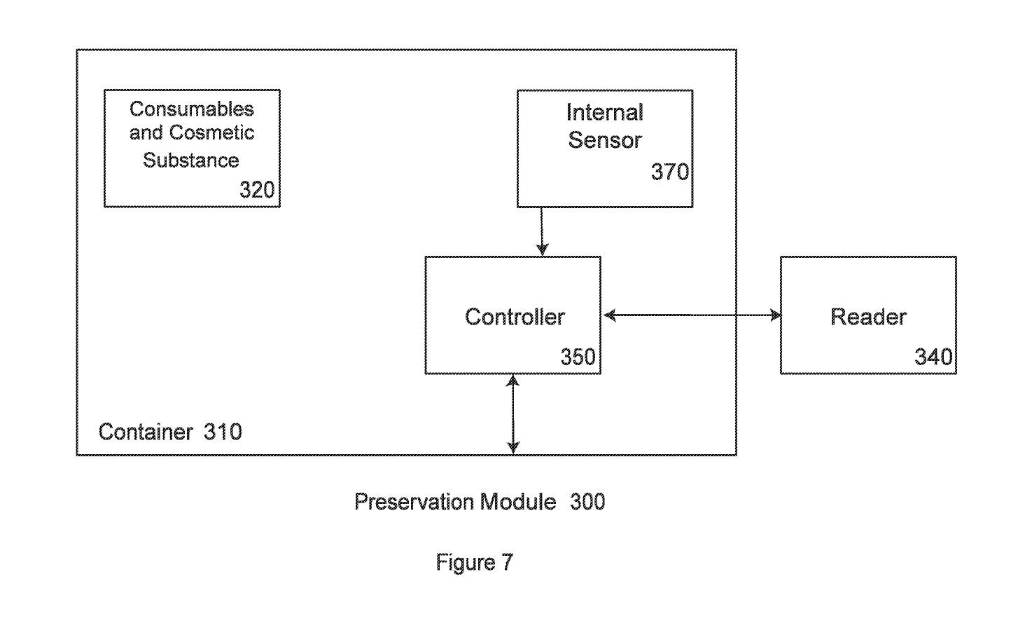

FIG. 7 shows embodiment of preservation system 300 wherein container 310 contains consumables and cosmetic substance 320 as well as controller 350. Controller 350 is connected to internal sensor 370 located either inside, or on the surface of, container 310, such that internal sensor 370 can obtain information regarding the environment internal to container 310. Controller 350 and internal sensor 370 can take the form of electronic components such as a micro-controller and an electronic sensor, or any other known sensor/controller formats. For example, the controller-sensor combination may be chemical or organic materials which perform the same function, such as a liquid crystal sensor/display. It is understood that the connection between internal sensor 370 and reader 340 may be by any connection methodologies known to one skilled in the art, including, but not limited to, physical connection, electronic connection, optical connection, wireless connection, or other near-field technologies. Such methodologies could be accomplished by local or remote interrogation of information sensor 370 by reader 340, or may be accomplished by transmission, such as by sensor 370 transmitting data to a remote or local reader 340, or any combination thereof. The controller-sensor combination may also be chemical or organic materials which perform the same function, such as a liquid crystal sensor/display.

When the shipper or user of container 310 desires information from internal sensor 370 the shipper or user can use reader 340 to query controller 350. In the electronic component embodiment, reader 340 could be a user interface device such as a computer which can be electronically or wirelessly connected to controller 350.

In an additional embodiment, controller 350 can modify the operation of container 310 so as modify the preservation capabilities of container 310, so as to favorably influence a ΔE of the consumables and cosmetic substance. For example, if the interior environment of container 310 would adversely affect the consumables and cosmetic substance 320, controller 350 could adaptively modify the internal environment of container 310 to better preserve the consumables and cosmetic substance. If consumables and cosmetic substance needs to be kept within a certain temperature range to preserve its efficacy properties, and the internal sensor 370 provide internal temperature information to controller 350, controller 350 could adaptively modify container 310 so as to maintain consumables and cosmetic substance 320 within the required temperature range.

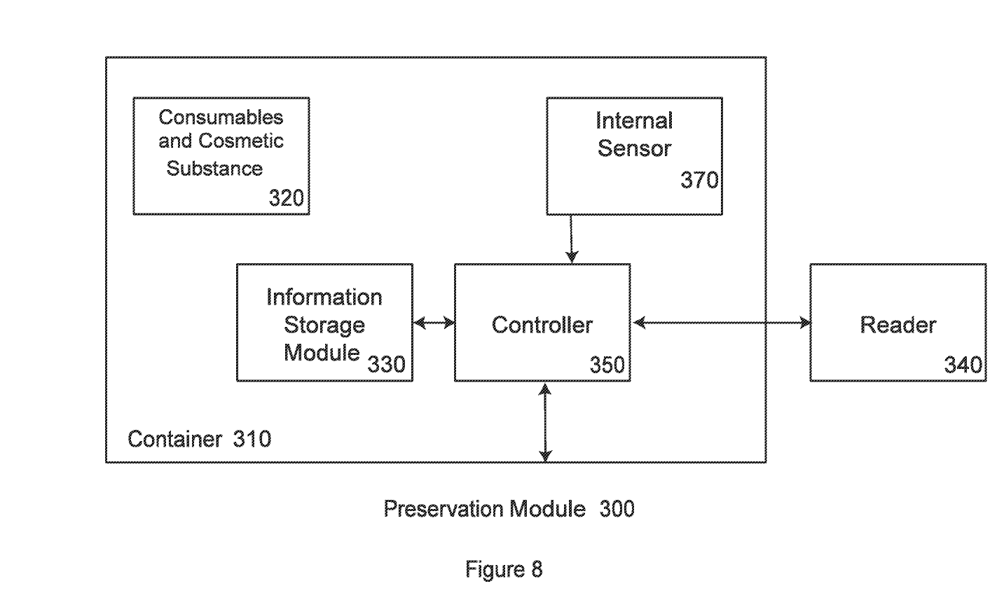

In FIG. 8, preservation system 300 includes container 310 which contains consumables and cosmetic substance 320, controller 350, and information storage module 330. Internal sensor 370 is positioned such that it can provide information on the internal environment to container 310. Information from the internal sensor and information storage module can be retrieved by connecting reader 340 to controller 350, which might be accomplished locally or remotely.

In this embodiment, information regarding the internal environment sensed by internal sensor 370 and provided to controller 350 can be stored in information storage module 330. This storage of internal environment can be used to record a history the internal environment container 310 has been subjected to. This would allow the shipper or user of container 310 to understand the internal environment the container has been subjected to during the time it has preserved the consumables and cosmetic substance. Such information can be used to determine any number of ΔE values of the consumables and cosmetic substance and if the consumables and cosmetic substance is no longer safe for consumption or one or more of its ΔE values has been degraded such that the consumables and cosmetic substance is no longer in an optimal state or has expired. Additionally, the user of the consumables and cosmetic substance could modify its transformation, dispensing, or consumption according to any changes, or ΔEs, that may have occurred because of the internal conditions of the container.

Additionally, in this embodiment, information storage module 330 could contain other information regarding the consumables and cosmetic substance, including creation information, identification information, and/or prior preservation or transformation information.

In an additional embodiment, controller 350 can modify the operation of container 310 so as modify the preservation capabilities of container 310. For example, if the internal environment of container 310 would adversely affect the consumables and cosmetic substance 320, controller 350 could adjust the internal environment of container 310, so as to favorably influence a ΔE of the consumables and cosmetic substance. For example, if the internal environment of container 310 would adversely affect the consumables and cosmetic substance 320, controller 350 could adaptively modify the internal environment of container 310 responsive to the data measured by sensor 370, to better preserve the consumables and cosmetic substance. Controller 350 can analyze the historic information from internal sensor 370, stored in information storage module 330 to determine any long-term internal environmental conditions. If consumables and cosmetic substance 320 needs to be kept within a certain temperature range to preserve its efficacy properties, and the internal sensor 370 provides internal temperature information to controller 350, controller 350 could adaptively modify container 310 so as to maintain consumables and cosmetic substance 320 within the required temperature range.

Information in the information storage module 330 might include identification information, information regarding prior preservation or transformation of the consumables and cosmetic substance 320, and other historic information. A shipper, or user, of container 310 can operatively connect to information storage module 330 using reader 340 to retrieve information stored therein. In an alternate embodiment reader 340 can also write to information storage module 330. In this embodiment, information regarding the container and/or consumables and cosmetic substance 320 can be modified or added to information storage module 330 by the user or shipper.

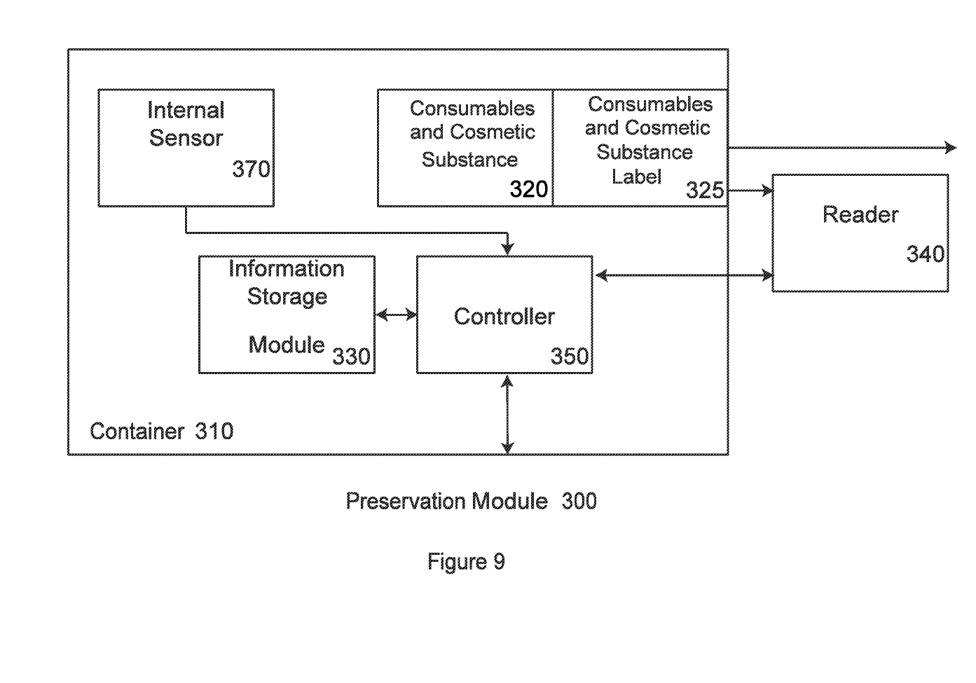

FIG. 9 shows an alternate embodiment of the present invention. Preservation module 300 includes container 310 which contains consumables and cosmetic substance 320, consumables and cosmetic substance label 325, controller 350, and information storage module 330. Internal sensor 370 is positioned such that it can provide information on the internal environment to container 310. Information from the internal sensor and information storage module can be locally or remotely retrieved by connecting reader 340 to controller 350. In one embodiment, this could be accomplished locally by physical connection to container 310. Consumables and cosmetic substance label 325 is attached to consumables and cosmetic substance 320 so as to sense, measure, and/or indicate the current efficacy state of consumables and cosmetic substance 320. Consumables and cosmetic substance label 325 can be read by reader 340. Consumables and cosmetic substance label 325 could be a material/chemical tag that, through a physical reaction with the surface of consumables and cosmetic substance 320, provides information regarding the efficacy state of the consumables and cosmetic substance, or information regarding changes in the efficacy values of the consumables and cosmetic substance, including where consumables and cosmetic substance 320 is in its life cycle, particularly the time during which its efficacy value is useful. As an example, this label/tag could change color as a consumables and cosmetic substance's efficacy values change over a change in condition that the consumables and cosmetic substance experiences. It could also indicate if it detects traces of pesticides, hormones, allergens, harmful or dangerous bacteria, or any other adulterating substances.

In this embodiment, information regarding the internal environment sensed by internal sensor 370 and provided to controller 350 can be stored in information storage module 330. This storage of internal environment can be used to record a history the internal environment container 310 has been subjected to. This would allow the shipper or user of container 310 to understand the internal environment the container has been subjected to during the time it has preserved the consumables and cosmetic substance. Such information can be used to determine any number of ΔE values for the consumables and cosmetic substance, including if the consumables and cosmetic substance is no longer safe for consumption or if a ΔE value has been degraded such that the consumables and cosmetic substance is no longer in an optimal state or has expired. Additionally, the user of the consumables and cosmetic substance could adaptively modify its transformation, dispensing, or consumption according to any changes, or ΔEs, that may have occurred because of the internal conditions of the container.

Additionally, in this embodiment, information storage module 330 could contain other information regarding the consumables and cosmetic substance, including creation information, identification information, and/or prior preservation or transformation information.

In an additional embodiment, controller 350 can modify the operation of container 310 so as modify the preservation capabilities of container 310, so as to favorably influence a ΔE of the consumables and cosmetic substance. For example, if the internal environment of container 310 would adversely affect the consumables and cosmetic substance 320, controller 350 could adaptively modify the internal environment of container 310, responsive to the information regarding the internal environment, to better preserve the consumables and cosmetic substance. Controller 350 can analyze the historic information collected by internal sensor 370 and stored in information storage module 330 to determine any long-term internal conditions environmental If consumables and cosmetic substance needs to be kept within a certain temperature range to preserve its efficacy properties, and the internal sensor 370 provide internal temperature information to controller 350, controller 350 could adaptively modify container 310 so as to maintain consumables and cosmetic substance 320 within the required temperature range.

Information in the information storage module 330 might include identification information, information regarding prior preservation or transformation of the consumables and cosmetic substance 320, and other historic information. A shipper, or user, of container 310 can operatively connect to information storage module 330 using reader 340 to retrieve information stored therein. Additionally, such a shipper, or user, of container 310 can obtain information from consumables and cosmetic substance label 325, either through direct observation or through reader 340. In an alternate embodiment reader 340 can also write to information storage module 330. In this embodiment, information regarding the container and/or consumables and cosmetic substance 320 can be modified or added to information storage module 330 by the user or shipper.

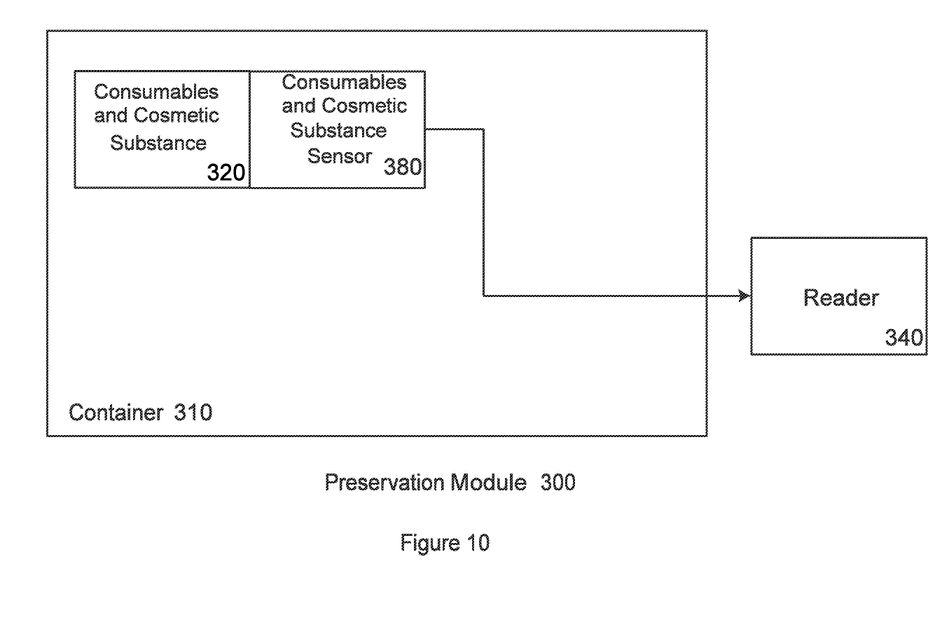

FIG. 10 shows an embodiment of preservation module 300 wherein container 310 contains consumables and cosmetic substance 320 as well as consumables and cosmetic substance sensor 380 in contact, or close proximity, with consumables and cosmetic substance 320, such that consumables and cosmetic substance sensor 380 can obtain information regarding the consumables and cosmetic substance 320 in container 310. Consumables and cosmetic substance sensor 380 and reader 340 can take the form of electronic components such as an electronic sensor and electronic display, or any other known sensor/reader formats. For example, the reader-sensor combination may be chemical or organic materials which perform the same function, such as a liquid crystal sensor/display. It is understood that the connection between consumables and cosmetic substance sensor 380 and reader 340 may be by any connection methodologies known to one skilled in the art, including, but not limited to, physical connection, electronic connection, optical connection, wireless connection, or other near-field technologies. Such methodologies could be accomplished by local or remote interrogation of sensor 380 by reader 340, or may be accomplished by transmission, such as by sensor 380 transmitting data to a remote or local reader 340.

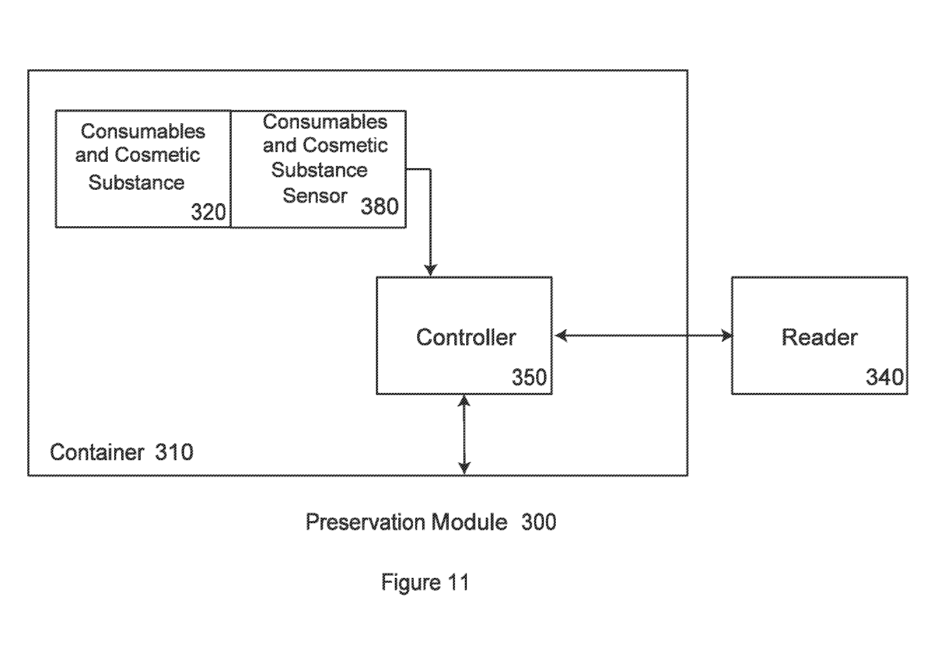

FIG. 11 shows embodiment of preservation module 300 wherein container 310 contains consumables and cosmetic substance 320 as well as controller 350. Controller 350 is connected to consumables and cosmetic substance sensor 380. Consumables and cosmetic substance sensor 380 and controller 350 can take the form of electronic components such as an electronic sensor and electronic display, or any other known sensor/controller formats. For example, the sensor/controller combination may be chemical or organic materials which perform the same function, such as a liquid crystal sensor/display. It is understood that the connection between controller 350 and reader 340 may be by any connection methodologies known to one skilled in the art, including, but not limited to, physical connection, electronic connection, optical connection, wireless connection, or other near-field technologies. Such methodologies could be accomplished by local or remote interrogation of controller 350 by reader 340, or may be accomplished by transmission, such as by controller 350 transmitting data to a remote or local reader 340.

When the shipper or user of container 310 desires information from consumables and cosmetic substance sensor 380 the shipper or user can use reader 340 to query consumables and controller 350. In the electronic component embodiment, reader 340 could be a user interface device such as a computer which can be electronically connected to controller 350.

In an additional embodiment, controller 350 can modify the operation of container 310 so as modify the preservation capabilities of container 310, so as to favorably influence a ΔE of the consumables and cosmetic substance. For example, if the data provide to controller 350 by sensor 380 indicates an undesirable or unexpected ΔE value, the interior environment of container 310, controller 350 could adaptively adjust the consumables and cosmetic substance environment of container 310 responsive to the data provided by sensor 380 to better preserve the consumables and cosmetic substance. If a rate of change in an efficacy value of the consumables and cosmetic substance needs to be kept within a certain range to preserve its efficacy properties, and the consumables and cosmetic substance sensor 380 provide consumables and cosmetic substance information to controller 350, controller 350 could adaptively modify container 310 so as to maintain consumables and cosmetic substance 320 within the required rate of change in the efficacy value.

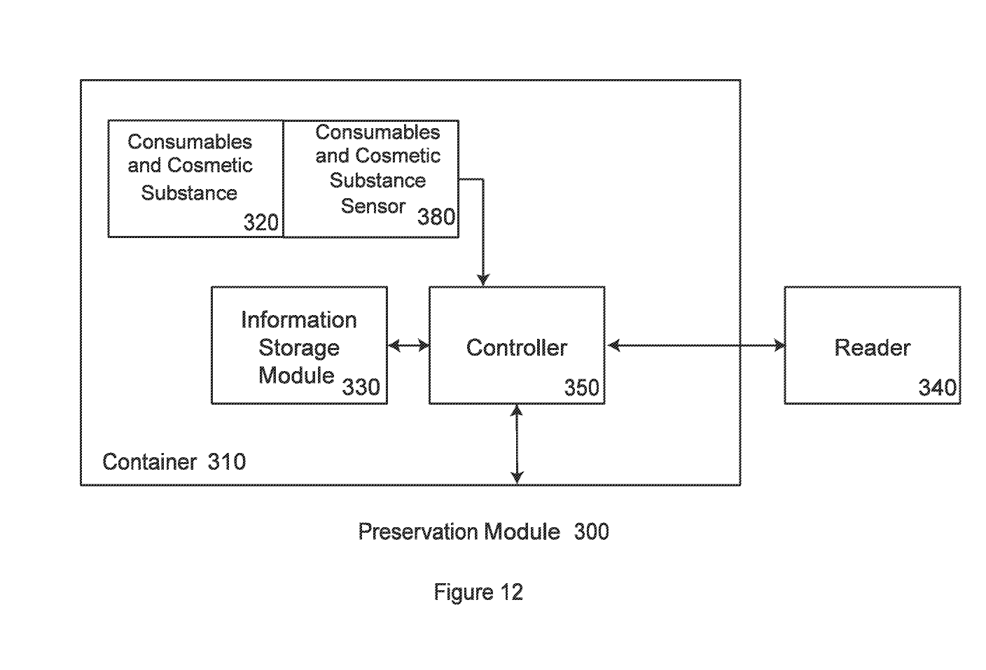

In FIG. 12, preservation module 300 includes container 310 which contains consumables and cosmetic substance 320, controller 350, and information storage module 330. Consumables and cosmetic substance sensor 380 is positioned such that it can provide information on the consumables and cosmetic substance in container 310. Information from the consumables and cosmetic substance sensor 380 and information storage module can be retrieved by connecting reader 340 to controller 350.

In this embodiment, information regarding the consumables and cosmetic substance sensed by consumables and cosmetic substance sensor 380, and provided to controller 350, can be stored in information storage module 330. This storage of consumables and cosmetic substance information can be used to record a history the consumables and cosmetic substance. This would allow the shipper or user of container 310 to understand the consumables and cosmetic substance during the time it has been. Such information can be used to determine any number of ΔE values of the consumables and cosmetic substance and if the consumables and cosmetic substance is no longer safe for consumption or if its ΔE has been degraded such that the consumables and cosmetic substance is no longer in an optimal state or has expired. Additionally, the user of the consumables and cosmetic substance could modify its transformation, dispensing, or consumption according to any changes, or ΔEs, that may have occurred.

Additionally, in this embodiment, information storage module 330 could contain other information regarding the consumables and cosmetic substance, including creation information, identification information, and/or prior preservation and transformation information.

In an additional embodiment, controller 350 can modify the operation of container 310 so as modify the preservation capabilities of container 310. For example, if the consumables and cosmetic substance 320 is being adversely affected, such as an undesirable rate of change in an efficacy value, controller 350 could adjust the container 310 responsive to the efficacy data to better preserve the consumables and cosmetic substance. Controller 350 can analyze the historic information from consumables and cosmetic substance sensor 380 stored in information storage module 330 to determine how the consumables and cosmetic substance's efficacy properties evolved during its preservation. If the rate of change in an efficacy value of the consumables and cosmetic substance needs to be kept within a certain range to preserve its efficacy properties, and the consumables and cosmetic substance sensor 380 provide consumables and cosmetic substance information to controller 350, controller 350 could adaptively modify container 310 so as to maintain consumables and cosmetic substance 320 within the required range for rate of change of the efficacy value.

Information in the information storage module 320 might include identification information, information regarding prior preservation or transformation of the consumables and cosmetic substance 320, and other historic information. A shipper, or user, of container 310 can operatively connect to information storage module 330 using reader 340 to retrieve information stored therein. In an alternate embodiment reader 340 can also write to information storage module 330. In this embodiment, information regarding the container and/or consumables and cosmetic substance 320 can be modified or added to information storage module 330 by the user or shipper.

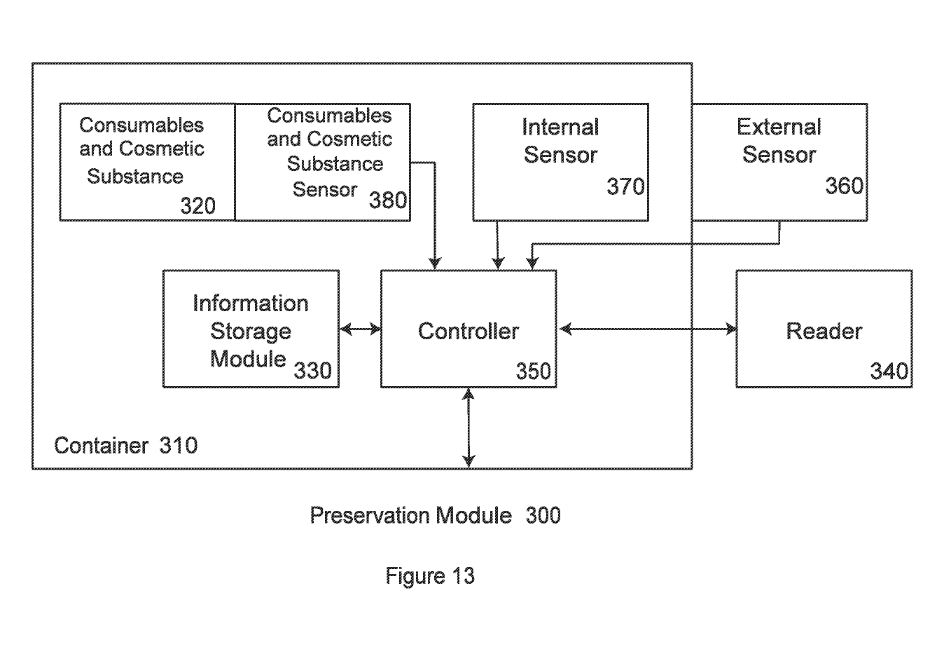

FIG. 13 shows the preferred embodiment of preservation module 300. Within container 310 is consumables and cosmetic substance 320, consumables and cosmetic substance sensor 380, internal sensor 370, information storage module 330, and controller 350. External sensor 360 is located outside or on the surface of container 310. In operation, controller 350 receives information from consumables and cosmetic substance sensor 380, internal sensor 370, and external sensor 360. Additionally, controller 350 can store the information received from the three sensors in in information storage module 330. Controller 350 can retrieve such stored information and transmit it to reader 340. Reader 340 can also transmit instructions to controller 350.

Controller 350 is operably connected to container 310 so as to use the information obtained from the sensors and/or information stored in the information storage module to modify the operation of container 310 to affect the state of consumables and cosmetic substance 320, that is, to favorably influence a ΔE for the consumables and cosmetic substance. Additionally, storage module 330 could contain information regarding consumables and cosmetic substance 320 as to its identity, creation information and/or prior preservation or transformation information. This historic information could also be used in modifying the operation of container 310 in its preservation of consumables and cosmetic substance 320.

As an example, consumables and cosmetic substance 320 is being shipped in container 310 to a distribution warehouse. Container 310 is capable of controlling its internal temperature, humidity, and the level of certain gasses within the container. Creation information as to consumables and cosmetic substance 320 is placed in information storage module 330 prior to shipment. During shipment, external sensor 360 measures the temperature and humidity outside container 310. This information is received by controller 350 and stored in information storage module 330. Controller 350 also receives information on the internal environment within container 310 from internal sensor 370 and stores this information in information storage module 330. This information includes the internal temperature, humidity, and certain gas levels within container 310. Finally, consumables and cosmetic substance sensor 380, which is attached to the surface of the consumables and cosmetic substance, provides information as to the state of the consumables and cosmetic substance to controller 350. This information could include surface temperature, surface humidity, gasses being emitted, and surface chemicals. At any time during its shipment and delivery to the distribution warehouse, reader 340 can be used to locally or remotely retrieve both current information and historic information stored within information storage module 330.

During shipment, container 310 modifies its internal conditions according to instructions provided by controller 350. Controller 350 contains instructions as to how to preserve an efficacy value of the consumables and cosmetic substance using information stored in information storage module 330 about the creation of the consumables and cosmetic substance, as well as historical information received from the three sensors, as well as current information being received from the three sensors. In this manner, preservation module 300 can preserve and optimize and minimize degradation of the efficacy value of the consumables and cosmetic substance. In other words, preservation module 300 can operate in a way to favorably influence changes in efficacy values, ΔEs, of the consumables and cosmetic substance while it is being shipped and stored.

It will be understood that subsets of the embodiment described herein can operate to achieve the goals stated herein. In one embodiment, consumables and cosmetic substance sensor 380, internal sensor 370, external sensor 360, information storage module 330, controller 350, reader 340, and parts of container 310 are each electrical or electromechanical devices which perform each of the indicated functions. However, it is possible for some or all of these functions to be done using chemical and/or organic compounds, or any sensing technologies known to one having skill in the art. For example, a specifically designed plastic wrap for some consumables and cosmetic substances can sense the exterior conditions of the package, the interior conditions of the package, and control gas flow through its surface so as to better preserve an efficacy value of the consumables and cosmetic substance.

Unless the context clearly requires otherwise, throughout the description and the claims, the words “comprise,” “comprising,” and the like are to be construed in an inclusive sense (i.e., to say, in the sense of “including, but not limited to”), as opposed to an exclusive or exhaustive sense. As used herein, the terms “connected,” “coupled,” or any variant thereof means any connection or coupling, either direct or indirect, between two or more elements. Such a coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the invention is not intended to be exhaustive or to limit the invention to the precise form disclosed above. While specific examples for the invention are described above for illustrative purposes, various equivalent modifications are possible within the scope of the invention, as those skilled in the relevant art will recognize. While processes or blocks are presented in a given order in this application, alternative implementations may perform routines having steps performed in a different order, or employ systems having blocks in a different order. Some processes or blocks may be deleted, moved, added, subdivided, combined, and/or modified to provide alternative or sub-combinations. Also, while processes or blocks are at times shown as being performed in series, these processes or blocks may instead be performed or implemented in parallel, or may be performed at different times. Further any specific numbers noted herein are only examples. It is understood that alternative implementations may employ differing values or ranges.

The various illustrations and teachings provided herein can also be applied to systems other than the system described above. The elements and acts of the various examples described above can be combined to provide further implementations of the invention.

Any patents and applications and other references noted above, including any that may be listed in accompanying filing papers, are incorporated herein by reference. Aspects of the invention can be modified, if necessary, to employ the systems, functions, and concepts included in such references to provide further implementations of the invention.

These and other changes can be made to the invention in light of the above Detailed Description. While the above description describes certain examples of the invention, and describes the best mode contemplated, no matter how detailed the above appears in text, the invention can be practiced in many ways. Details of the system may vary considerably in its specific implementation, while still being encompassed by the invention disclosed herein. As noted above, particular terminology used when describing certain features or aspects of the invention should not be taken to imply that the terminology is being redefined herein to be restricted to any specific characteristics, features, or aspects of the invention with which that terminology is associated. In general, the terms used in the following claims should not be construed to limit the invention to the specific examples disclosed in the specification, unless the above Detailed Description section explicitly defines such terms. Accordingly, the actual scope of the invention encompasses not only the disclosed examples, but also all equivalent ways of practicing or implementing the invention under the claims.

While certain aspects of the invention are presented below in certain claim forms, the applicant contemplates the various aspects of the invention in any number of claim forms. For example, while only one aspect of the invention is recited as a means-plus-function claim under 35 U.S.C. §112, sixth paragraph, other aspects may likewise be embodied as a means-plus-function claim, or in other forms, such as being embodied in a computer-readable medium. Any claims intended to be treated under 35 U.S.C. §112, ¶ 6 will begin with the words “means for.” Accordingly, the applicant reserves the right to add additional claims after filing the application to pursue such additional claim forms for other aspects of the invention.

1. A preservation system for consumables and cosmetic substances comprising:
an adaptive preserver for adaptively preserving a consumables and cosmetic substance; and
a sensor for sensing at least one of an internal attribute and external attribute of the adaptive preserver;
wherein the adaptive preserver adaptively preserves said consumables and cosmetic substance in response to said at least one of an internal attribute and external attribute of the adaptive preserver.

2. A preservation system for consumables and cosmetic substances according to claim 1 further comprising attribute storage for storing said at least one of an internal attribute and external attribute.

3. A preservation system for consumables and cosmetic substances according to claim 1 further comprising a transmitter for transmitting said at least one of an internal attribute and external attribute.

4. A preservation system for consumables and cosmetic substances according to claim 1 further comprising a reader for reading said at least one of an internal attribute and external attribute

5. A preservation system for consumables and cosmetic substances comprising:
an adaptive preserver for adaptively preserving a consumables and cosmetic substance; and
a sensor for sensing an attribute of the consumables and cosmetic substance;
wherein the adaptive preserver adaptively preserves said consumables and cosmetic substance in response to the attribute of the consumables and cosmetic substance.

6. A preservation system for consumables and cosmetic substances according to claim 5 further comprising attribute storage for storing the attribute.

7. A preservation system for consumables and cosmetic substances according to claim 5 further comprising a transmitter for transmitting the attribute.

8. A preservation system for consumables and cosmetic substances according to claim 5, wherein said adaptive preserver comprises a container which adapts at least one of its chemical, biological, electrical, thermal, humidity, and mechanical properties.

9. A preservation system for consumables and cosmetic substances according to claim 5, wherein said sensor comprises at least one of a chemical, biological, electrical, and mechanical sensors.

10. A preservation system for consumables and cosmetic substances according to claim 5, wherein said attribute storage comprises at least one of a computer and a database.

11. A consumables and cosmetic substance tracking system for tracking dynamically changing values of consumables and cosmetic substances comprising:
an adaptive preserver for adaptively preserving a consumables and cosmetic substance responsive to a change in an efficacy value of a consumables and cosmetic substance; and
a sensor for sensing the change in an efficacy value of the consumables and cosmetic substance; and
attribute storage for storing the change in an efficacy value of the consumables and cosmetic substance.

12. A consumables and cosmetic substance tracking system for tracking dynamically changing values of consumables and cosmetic substances according to claim 11 wherein the adaptive preserver adaptively preserves the consumables and cosmetic substance so as to maintain, or minimize degradation of the efficacy value of the consumables and cosmetic substance.

13. A consumables and cosmetic substance tracking system for tracking dynamically changing values of consumables and cosmetic substances according to claim 11, wherein said adaptive preserver comprises a container which adapts at least one of its chemical, biological, electrical, thermal, humidity, and mechanical properties in response to the change in efficacy value.

14. A consumables and cosmetic substance tracking system for tracking dynamically changing values of consumables and cosmetic substances according to claim 11, wherein said sensor comprises at least one of a chemical, biological, electrical, optical, and mechanical sensors.

15. A consumables and cosmetic substance tracking system for tracking dynamically changing values of consumables and cosmetic substances according to claim 11, wherein said attribute storage comprises at least one of a computer or a database.

16. A method of dynamically ascertaining an expiration date for consumables and cosmetic substances comprising the steps of:
measuring a dynamically changing condition associated with a consumables and cosmetic substance; and
comparing said measured condition to known conditions associated with similar consumables and cosmetic substances; and
correlating a residual efficacy value of the consumables and cosmetic substance with the measured efficacy value of the similar consumables and cosmetic substances; and
determining if the correlated residual efficacy value of the consumables and cosmetic substance has expired.

17. The method of dynamically ascertaining an expiration date for consumables and cosmetic substances according to claim 16 wherein the measured condition is an attribute of the consumables and cosmetic substance's environment.

18. The method of dynamically ascertaining an expiration date for consumables and cosmetic substances according to claim 16 wherein the measured condition is an attribute of the consumables and cosmetic substance's packaging.

19. The method of dynamically ascertaining an expiration date for consumables and cosmetic substances according to claim 16 wherein the known conditions associated with similar consumables and cosmetic substances are based on at least one of experimentation and algorithm.
